(19) United States
(12) Patent Application Publication
(10) Pub. No.: US 2012/0078094 A1
(43) Pub. Date: Mar. 29, 2012

(54) Ultrasound-Guided Ablation Method and Ultrasound-Guided Ablation System

(75) Inventors: Kenichi NISHINA, Tokyo (JP); Takeharu NAKAZATO, Tokyo (JP); Hideyuki KASAHARA, Tokyo (JP); Norihiro YAMADA, Tokyo (JP); Yukihiko SAWADA, Yoshikawa-shi (JP); Shuji OTANI, Tokyo (JP); Takahiro ECHIZENYA, Tokyo (JP)

(73) Assignee: OLYMPUS MEDICAL SYSTEMS CORP., Tokyo (JP)

(21) Appl. No.: 13/315,876

(22) Filed: Dec. 9, 2011

Related U.S. Application Data
(62) Division of application No. 12/206,452, filed on Sep. 8, 2008.

(51) Int. Cl. A61B 8/00

(52) U.S. Cl. 600/431

(57) ABSTRACT

An ultrasound-guided ablation method captures an objective area to be ablated in an ultrasound scan area of an ultrasound transducer and delineates the objective area on an ultrasound image; specifies an ablation target area to display the ablation target area with a margin necessary for ablating the objective area on the ultrasound image processed by an ultrasound observation device and displayed on a display device; ablates, by an ablation device, the ablation target area displayed on the ultrasound image; and checks, on the ultrasound image, that an ablated area ablated by the ablation device has reached the ablation target area displayed on the ultrasound image.

### CROSS-REFERENCE

This application is a Divisional of U.S. patent application Ser. No. 12/206,452 filed on Sep. 8, 2008, the contents of which are incorporated by this reference.

### BACKGROUND OF THE INVENTION

1. Field of the Invention

The present invention relates to an ultrasound-guided ablation method and an ultrasound-guided ablation system for treating an objective area such as tumor by ablation under the guidance of ultrasound.

2. Description of the Related Art

Conventionally, a treatment procedure has been known in which an ablation apparatus, such as a radio-frequency ablation device, a high-frequency ablation device, and a micro-wave ablation device, is punctured under the guidance of ultrasound from the body surface to liver tumor or the like to ablate an objective area such as a lesion.

In recent years, an ultrasound endoscope-guided ablation procedure is being studied as an example in which an ultrasound endoscope provided with a linear/convex transducer is used for performing the procedure.

In the ablation procedure, leaving a diseased tissue such as a tumor cell needs to be prevented. Therefore, it is desirable to ablate an area with a margin of, for example, 5 mm with respect to the objective area such as a lesion.

However, in an ultrasound image, the boundary between an ultrasound image of the objective area such as a lesion and an ultrasound image of surrounding tissues become unclear, because the protein of the ablated tissue is denatured. As a result, even if the ablation is performed with the margin of 5 mm, with how much margin the ablation has been actually performed may not be able to be determined from the ultrasound image after the ablation.

### SUMMARY OF THE INVENTION

An ultrasound-guided ablation method of the present invention comprises: capturing an objective area to be ablated in an ultrasound scan area of an ultrasound transducer and delineating the objective area on an ultrasound image; specifying an ablation target area to display the ablation target area with a margin necessary for ablating the objective area on the ultrasound image processed by an ultrasound observation device and displayed on a display device; ablating, by an ablation device, the ablation target area displayed on the ultrasound image; and checking, on the ultrasound image, that an ablated area ablated by the ablation device has reached the ablation target area displayed on the ultrasound image.

The above and other objects, features and advantages of the invention will become more clearly understood from the following description referring to the accompanying drawings.

### BRIEF DESCRIPTION OF THE DRAWINGS

FIGS. 1 to 16 illustrate a first embodiment;

FIG. 1 is a diagram for explaining a configuration of an ultrasound endoscope-guided ablation system;

FIG. 2 is a diagram for explaining a distal end rigid portion of an ultrasound endoscope and an ultrasound transducer arranged on the distal end rigid portion used in the present ultrasound endoscope-guided ablation system;

FIG. 3 is a perspective view for explaining a puncture needle that also serves as a marker placing device;

FIG. 4 is an enlarged sectional view of the part shown with IV in FIG. 3;

FIG. 5 is a diagram for explaining the ablation by the ultrasound endoscope-guided ablation system;

FIG. 6 is a diagram for explaining the placement of a marker near the center of an objective area;

FIG. 7 is a diagram for explaining the distance measurement from the marker to a peripheral border of the objective area;

FIG. 8 is a diagram for explaining the distance measurement from the marker in a different cross section of the objective area to the peripheral border of the objective area;

FIG. 9 is a diagram for explaining a spherical ablation target area set by adding an ablation margin to a maximum value of the measured distances from the marker to the peripheral border of the objective area and a state in which an electrode portion of an ablation device is arranged in the objective area and the ablation is performed under the guidance of the ultrasound endoscope;

FIGS. 10 to 12 are diagrams for explaining another ablation method of the ultrasound endoscope-guided ablation system;

FIG. 10 is a diagram for explaining an ablation target area set by placing a plurality of markers in different sizes to the outside of the peripheral border of the objective area and adding an ablation margin to the peripheral border of the objective area;

FIG. 11 is a diagram for explaining a plurality of markers and an ablation target area in a different cross section of the objective area;

FIG. 12 is a diagram for explaining a state in which the electrode portion of the ablation device is arranged in the objective area and the ablation is performed under the guidance of the ultrasound endoscope;

Another ablation method of the ultrasound endoscope-guided ablation system will be described with reference to FIGS. 13 to 17;

FIG. 13 is a diagram for explaining the placement of a plurality of markers with an ablation margin outside the peripheral border of the objective area;

FIG. 14 is a diagram for explaining a state in which a plurality of markers are placed with the ablation margin outside the peripheral border of the objective area;

FIG. 15 is a diagram for explaining a plurality of markers placed outside the peripheral border of the objective area in a cross section of a XV-XV line of FIG. 14;

FIG. 16A is a diagram for explaining a state in which the electrode portion of the ablation device is arranged in the objective area and the ablation is performed under the guidance of the ultrasound endoscope;

FIG. 16B is a diagram for explaining the ablation performed such that the ablated area reaches an ablation target area defined by a virtual line connecting the markers;

FIGS. 17 to 37 illustrate a second embodiment;

FIG. 17 is a diagram for explaining another configuration of the ultrasound endoscope-guided ablation system;

FIG. 18 is a diagram for explaining a distal end rigid portion of the ultrasound endoscope and an ultrasound transducer arranged in the distal end rigid portion used in the present ultrasound endoscope-guided ablation system;

FIG. 19 is a diagram for explaining a state in which the objective area is captured in an ultrasound scan area of the ultrasound transducer;

FIG. 20 is a diagram for explaining an ultrasound image delineating the objective area captured by the ultrasound transducer;

FIG. 21 is a diagram for explaining the acquisition of peripheral line data of the delineated objective area in the ultrasound image;

FIG. 22 is a diagram for explaining the acquisition of ablation target area data in which the ablation margin is added to the acquired peripheral line data of the objective area;

FIG. 23 is a diagram for explaining a state in which the electrode portion of the ablation device is arranged in the objective area and the ablation is performed under the guidance of the ultrasound endoscope;

FIG. 24 is a diagram for explaining the ablation performed while observing whether the ablated part has reached the ablation target area;

FIGS. 25 to 31 are diagrams for explaining another ablation method of the ultrasound endoscope-guided ablation system;

FIG. 25 is a diagram for explaining an ultrasound endoscope including a 2D array type ultrasound transducer;

FIG. 26 is a diagram for explaining a state in which the objective area is captured in an ultrasound scan area of the 2D array type ultrasound transducer;

FIG. 27 is a diagram for explaining two ultrasound images delineating the objective area captured by the 2D array type ultrasound transducer;

FIG. 28 is a diagram for explaining the acquisition of peripheral line data of the delineated objective areas on two ultrasound images;

FIG. 29 is a diagram for explaining the acquisition of ablation target area data in which the ablation margin is added to the peripheral line data of the acquired objective area;

FIG. 30 is a diagram for explaining a state in which the electrode portion of the ablation device is arranged in the objective area and the ablation is performed under the guidance of the ultrasound endoscope;

FIG. 31 is a diagram for explaining the ablation performed while observing whether the ablated part has reached the ablation target area;

FIGS. 32 to 37 are diagrams for explaining another ablation method of the ultrasound endoscope-guided ablation system;

FIG. 32 is a diagram for explaining a state in which the objective area is captured in the ultrasound scan area of the ultrasound transducer and the placement of the marker outside the peripheral border of the objective area;

FIG. 33 is a diagram for explaining the delineated objective area and a delineated marker captured on the ultrasound image;

FIG. 34 is a diagram for explaining the acquisition of peripheral line data of the delineated objective area and the delineated marker on the ultrasound image;

FIG. 35 is a diagram for explaining the acquisition of ablation target area data in which the ablation margin is added to the peripheral line data of the acquired objective area;

FIG. 36 is a diagram for explaining a state in which the electrode portion of the ablation device is arranged in the objective area and the ablation is performed under the guidance of the ultrasound endoscope;

FIG. 37 is a diagram for explaining the ablation performed while observing whether the ablated part has reached the ablation target area;

FIG. 38 is a diagram for explaining a configuration of the ultrasound endoscope for preventing the movement of the ultrasound transducer during an ablation procedure;

FIG. 39 is a diagram for explaining a configuration in which two ablation devices are arranged in the ultrasound endoscope to perform the ablation on a wide scale at a time;

FIG. 40 is a diagram for explaining a state in which the electrode portions of two ablation devices are arranged in the objective area and the ablation is performed under the guidance of the ultrasound endoscope;

FIG. 41A is a longitudinal sectional view for explaining a configuration example of the ultrasound endoscope capable of installing three ablation devices to perform the ablation on a wide scale at a time;

FIG. 41B is a diagram of the ultrasound endoscope of FIG. 41A as seen from the distal end side;

FIG. 42 is a diagram for explaining a state in which the electrode portions of three ablation devices are arranged in the objective area and the ablation is performed under the guidance of the ultrasound endoscope;

FIGS. 43 to 48 are diagrams for explaining operation from an operator operating the ultrasound endoscope to delineating the objective area on the ultrasound image;

FIG. 43 is a diagram for explaining a state in which a large vessel as a base point is captured in the ultrasound scan area of the ultrasound transducer;

FIG. 44 is a diagram for explaining the placement of a first marker after moving the ultrasound transducer from the base point into an objective area direction in an anatomical sense and capturing a first mark;

FIG. 45 is a diagram for explaining the placement of a second marker after moving the ultrasound transducer from a first marker placement point into an objective area direction in an anatomical sense and capturing a second mark;

FIG. 46 is a diagram for explaining the placement of a third marker, a state in which the objective area is captured in the ultrasound scan area, and a state in which a needle tube is punctured in the objective area after moving the ultrasound transducer from a second marker placement point into an objective area direction in an anatomical sense and capturing a third mark;

FIG. 47 is a diagram for explaining an example of markers that enable to easily determine, at a glance of the markers, the arranged order of the markers;

FIG. 48 is a diagram for explaining another example of markers that enable to easily determine, at a glance of the markers, the arranged order of the markers;

FIG. 49 is a diagram for explaining an example of rod-shaped ultrasound markers;

FIG. 50 is a diagram for explaining a rod-shaped ultrasound marker including a barb at the distal end;

FIG. 51 is a diagram for explaining a configuration example of a marker usable both as an ultrasound marker and as an endoscopic marker;

FIGS. 52 to 60 relate to a procedure that can preserve the function of a surrounding organ located near the objective area and that can surely ablate the objective area;

FIG. 52 is a diagram for explaining a configuration of a puncture needle with temperature sensor;

FIG. 53 is an enlarged view of the part shown with LIII of FIG. 52;

FIG. 54 is a cross-sectional view for explaining a configuration of a sensor cable connection part;

FIG. 55 is a diagram for explaining a state in which the ablation device and the puncture needle with temperature sensor are projected from two distal end openings included in the ultrasound endoscope;

FIG. 56 is a diagram for explaining a state in which the electrode portion of the ablation device is arranged at the objective area and a needlepoint of the puncture needle with temperature sensor is arranged between the objective area and the surrounding organ;

FIG. 57 is a diagram for explaining another configuration of the puncture needle with temperature sensor;

FIG. 58 is an enlarged view of the part shown with LVIII of FIG. 57;

FIG. 59 is a cross-sectional view for explaining a configuration of a stylet cap;

FIG. 60 is a diagram for explaining a state in which the electrode portion of the ablation device is arranged at the objective area and the stylet of the puncture needle with temperature sensor is arranged between the objective area and the surrounding organ;

FIGS. 61 to 63 are diagrams for explaining a puncture method enabling to determine whether the needlepoint has reached inside the objective area on the ultrasound image;

FIG. 61 is a diagram for explaining a state in which the needle tube is punctured into the objective area under the guidance of ultrasound;

FIG. 62 is a diagram for explaining a state in which the needlepoint is arranged in the objective area and an ultrasound contrast agent is injected into the objective area;

FIG. 63 is a diagram for explaining a state in which the ultrasound contrast agent is not injected into the objective area because the objective area is transformed and the needlepoint is not arranged in the objective area;

FIG. 64 is a diagram for explaining another puncture method enabling to determine whether the needlepoint has reached inside the objective area on the ultrasound image;

FIG. 64 is a diagram for explaining a state in which the needlepoint is arranged in the objective area and the marker is placed in the objective area; and

FIG. 65 is a diagram for explaining a state in which the marker is placed outside the peripheral border of the objective area because the needlepoint is not arranged in the objective area.

### DETAILED DESCRIPTION OF THE PREFERRED EMBODIMENTS

Preferred embodiments of the present invention will now be described with reference to the drawings.

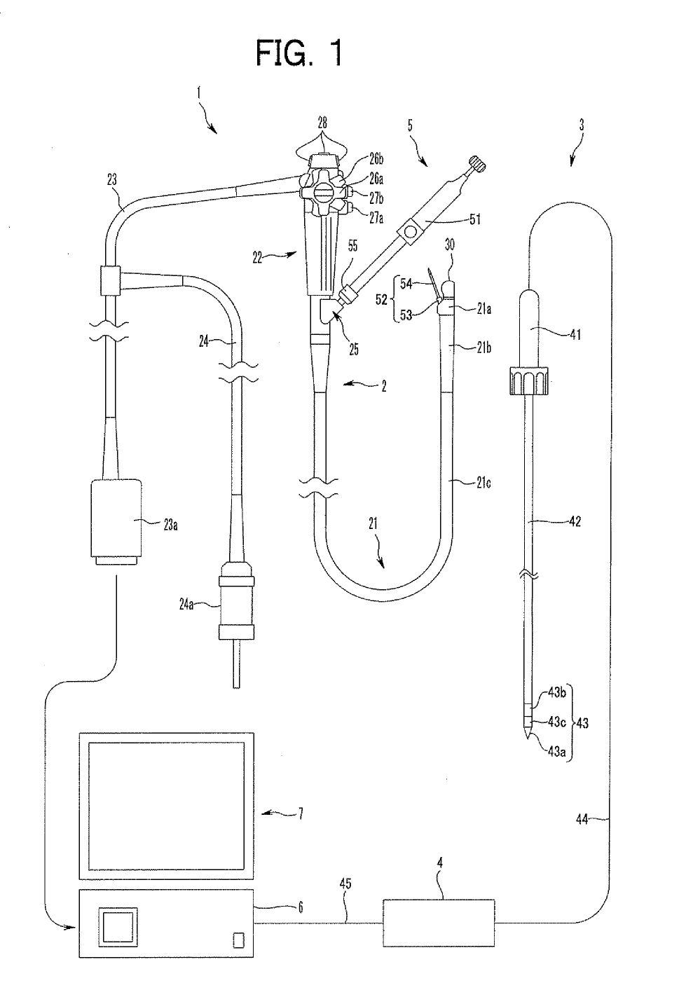

A first embodiment of the present invention will be described with reference to FIGS. 1 to 16B.

As shown in FIG. 1, an ultrasound endoscope-guided ablation system 1 of the present embodiment is mainly constituted by an ultrasound endoscope (hereinafter abbreviated as EUS) 2 that is a kind of endoscope, an ablation device 3, an ablation power source device (hereinafter abbreviated as power source device) 4, a puncture needle 5, an ultrasound observation device 6, and a display device 7.

The EUS 2 is mainly constituted by: an insertion portion 21 inserted into the body; an operation portion 22 located at a proximal end of the insertion portion 21; a universal code 23 extending from a side of the operation portion 22; and a light source cable 24 that is branched, for example, in the middle of the universal code 23.

An ultrasound connector 23a that can be attached to and detached from the ultrasound observation device 6 is arranged at a proximal end portion of the universal code 23. An endoscope connector 24a that can be attached to and detached from a light source device and a video processor device not shown is arranged at a proximal end portion of the light source cable 24.

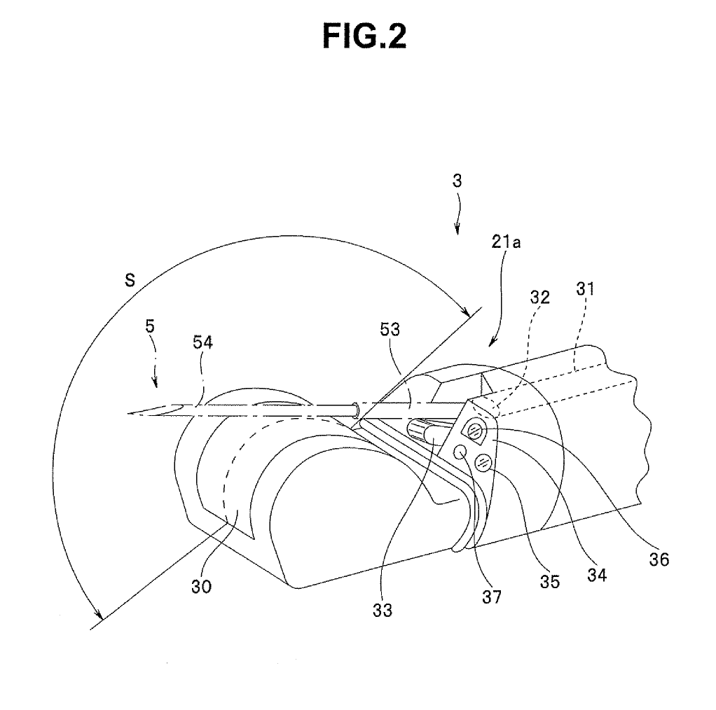

A treatment instrument insertion port 25 is arranged at a distal end side of the operation portion 22. The treatment instrument insertion port 25 is in communication with a treatment instrument channel (see reference numeral 31 of FIG. 2) arranged in the insertion portion 21. The treatment instrument insertion port 25 includes a ferrule, and a securing ring 56 arranged in a handle portion 51 of the puncture needle 5 and the like is connected to the ferrule. The securing ring 56 can be attached to and detached from the ferrule.

Reference numerals 26a and 26b denote bending operation knobs, reference numeral 27a denotes an air and water feeding button, reference numeral 27b denotes a suction button, and reference numeral 28 denotes a switch. The switch 28, for example, switches the display of the display device 7, provides a freeze instruction of a displayed image, provides a release instruction, or provides an ablation start/end instruction. Metal or the like is not arranged inside a treatment instrument channel 31 which is insulated from metals inside the endoscope.

The insertion portion 21 connects, in order from the distal end, a distal end rigid portion 21a, a bending portion 21b, and a flexible tube portion 21c. The bending portion 21b is configured to be actively bent, for example, in the vertical and horizontal directions by the operation of the bending operation knobs 26a and 26b. The flexible tube portion 21c is flexible.

As shown in FIG. 2, an electronically scanning ultrasound transducer 30 is arranged on the distal end side of the distal end rigid portion 21a. The ultrasound transducer 30 is, for example, a convex array and includes a plurality of ultrasound elements aligned inside.

A distal end opening 32 is arranged at the distal end of the treatment instrument channel 31 arranged at the distal end rigid portion 21a. A forceps raising table 33 is swingably arranged near the distal end opening 32. Swinging the forceps raising table 33, with a sheath 53 being mounted on the forceps raising table 33, enables to move a needle tube 54 to a desired position in a scanned surface S of the ultrasound transducer 30.

The distal end rigid portion 21a includes a slope portion 34 on the distal end side, and the slope portion 34 includes an illumination lens cover 35, an observation lens cover 36, and an air and water feeding nozzle 37.

The ablation device 3 is a radio-frequency ablation device and is mainly constituted by a grasping portion 41 grasped by the operator, a flexible tube portion 42, and an electrode portion 43. The grasping portion 41 is an insulating member and is cylindrical. The flexible tube portion 42 is constituted by an insulating flexible member and the proximal end of the flexible tube portion 42 is fixed to the grasping portion 41. The electrode portion 43 is arranged at a distal end surface of the flexible tube portion 42. A signal cable 44 extends from the grasping portion 41. The proximal end of the signal cable 44 is connected to the power source device 4. The power source device 4 is connected to the ultrasound observation device 6 through a connection cable 45 or to a video processor device not shown and is configured to transmit a signal of an ablation start/end instruction by the switch 28.

The electrode portion 43 arranged at the distal end surface of the flexible tube portion 42 is, for example, a bipolar electrode portion and is constituted by a first electrode 43a, a second electrode 43b, and an insulating portion 43c. The first electrode 43a and the second electrode 43b are annularly formed on the surface of the circumference of the flexible tube portion 42. The insulating portion 43c is arranged between the first electrode 43a and the second electrode 43b. The distal end of the signal cable 44 is inserted into the grasping portion 41 and into the flexible tube portion 42 and is connected to the first electrode 43a and the second electrode 43b.

The power source device 4 supplies high frequency radio waves with different polarities to the first electrode 43a and the second electrode 43b of the electrode portion 43 through the signal cable 44. When the power source device 4 supplies a radio frequency while the electrode portion 43 is inserted into body tissues, the electrode portion 43 is energized through body tissues between the first electrode 43a and the second electrode 43b and ablates the body tissues.

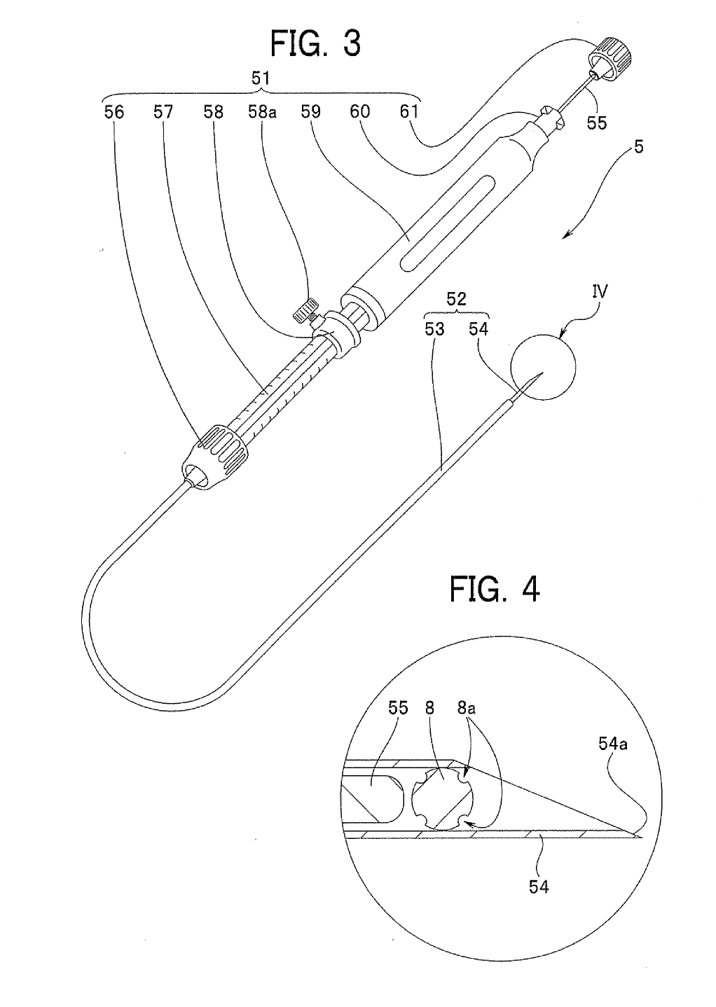

As shown in FIGS. 3 and 4, the puncture needle 5 includes the handle portion 51 and a channel insertion portion 52. The channel insertion portion 52 includes the sheath 53 and the needle tube 54. The channel insertion portion 52 is inserted into the treatment instrument channel 31 from the treatment instrument insertion port 25 and projects from the distal end opening 32 shown in FIG. 2.

In the present embodiment, the puncture needle 5 is an ablation target area specifying unit, the puncture needle 5 also serving as a marker placing device for placing a marker, which can be delineated on an ultrasound image, in a living body. Therefore, as shown in FIG. 4, a pusher 55, which can be inserted into and pulled out from the needle tube 54, is arranged in the needle tube 54. The pusher 55 is made of a metal rod such as stainless and nickel titanium alloy and is inserted into the needle tube 54 for pushing out the marker 8 arranged at the distal end of the needle tube 54 outside the needle tube 54.

The marker 8 is made of magnesium alloy that is absorbed in the living body in the course of time and is, for example, spherical. A plurality of ultrasound reflecting surfaces 8a are formed on the outer surface of the marker 8. The ultrasound reflecting surfaces 8a are, for example, circular concave portions.

The ultrasound reflecting surfaces 8a are not limited to be circular concave portions, but may also be annular concave portions including circular convex portions at the center. The marker 8 may also be made of biocompatible metal such as gold and titanium.

The needle tube 54 is inserted into the sheath 53 and is movable back and forth with respect to the sheath 53. The needle tube 54 is formed of a metal pipe such as a stainless pipe and a nickel titanium pipe. A sharp blade portion 54a is formed at the distal end of the needle tube 54.

The handle portion 51 includes, for example, in order from the distal end, a securing ring 56, an operation portion main body 57, a stopper member 58, a needle slider 59, a ferrule portion 60, and a pusher knob 61.

The proximal end portion of the pusher 55 is integrally fixed to the pusher knob 61 by, for example, gluing. The proximal end portion of the needle tube 54 is integrally fixed to the ferrule portion 60 by gluing or the like. The proximal end portion of the sheath 53 is integrally fixed to a predetermined location of the proximal end portion of the operation portion main body 57 by gluing or the like.

The stopper member 58 includes a fixing screw 58a. The stopper member 58 can adjust the position with respect to the operation portion main body 57 as necessary, with the fixing screw 58a being loosened. A projected length of the needle tube 54 from the distal end of the sheath 53 is adjusted by adjusting the position of the stopper member 58.

The flexible tube portion 42 of the ablation device 3 can be inserted into the needle tube 54.

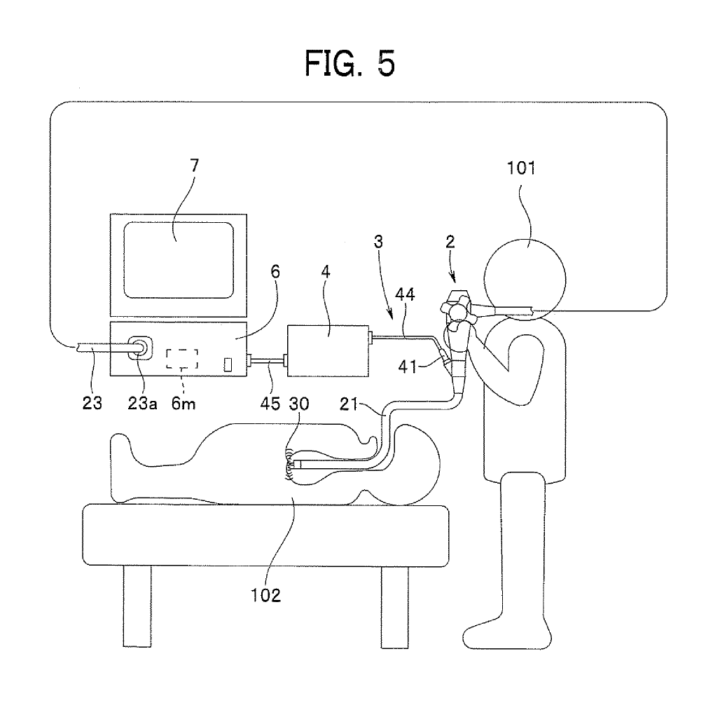

An ablation method of the ultrasound endoscope-guided ablation system 1 configured as described above will be described with reference to FIGS. 5 to 9.

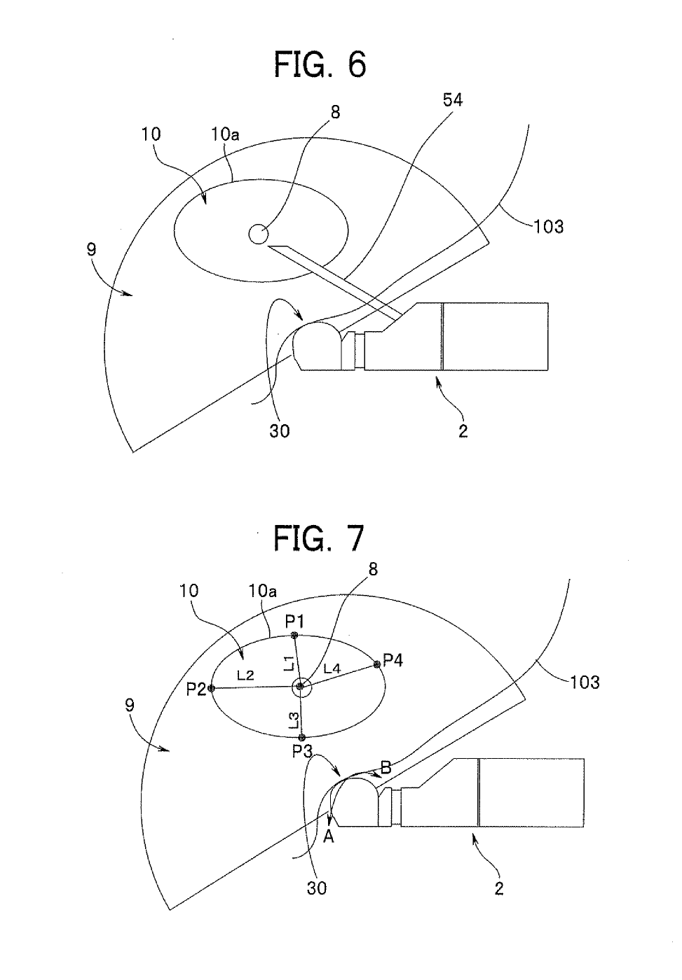

As shown in FIG. 5, an operator 101 inserts the insertion portion 21 of the EUS 2 into the body of a patient 102 through, for example, the mouth, observes an endoscopic image displayed on the display device 7, and inserts the ultrasound transducer 30 near the objective area. The operator then brings the ultrasound transducer 30 into contact with a lumen wall 103, as shown in FIG. 6.

The operator 101 then displays the ultrasound image on the display device 7 and, as shown in FIG. 6, captures an objective area 10, such as cancer, at a desired location in an ultrasound scan area 9.

The operator 101 then punctures the puncture needle 5 as a marker placing device under the guidance of the ultrasound endoscope such that the needle tube 54 is located near the center of the objective area 10. Subsequently, the operator 101 pushes in the pusher knob 61 to move the pusher 55 to place the marker 8 arranged at the distal end portion of the needle tube 54 into the objective area 10.

After checking the placement of the marker 8 to the objective area 10 based on the ultrasound image, the operator asks a medical personnel (hereinafter referred to as staff) to measure a distance from the marker 8 to an objective area peripheral border 10a.

Consequently, the staff selects, for example, four points P1, P2, P3, and P4 on the delineated objective area peripheral border 10a on the ultrasound image as shown in FIG. 7 and measures distances from the delineated marker 8. Reference numerals L1, L2, L3, and L4 of FIG. 7 denote the distances from the marker 8 as a base point to the points P1, 2, 3, and 4.

The operator then performs a hand-side operation to move the distal end side of the EUS 2 in an arrow A direction or in an arrow B direction of FIG. 7 to thereby move the ultrasound transducer 30. Consequently, an ultrasound image of a different cross section of the objective area 10 is delineated on the display device 7.

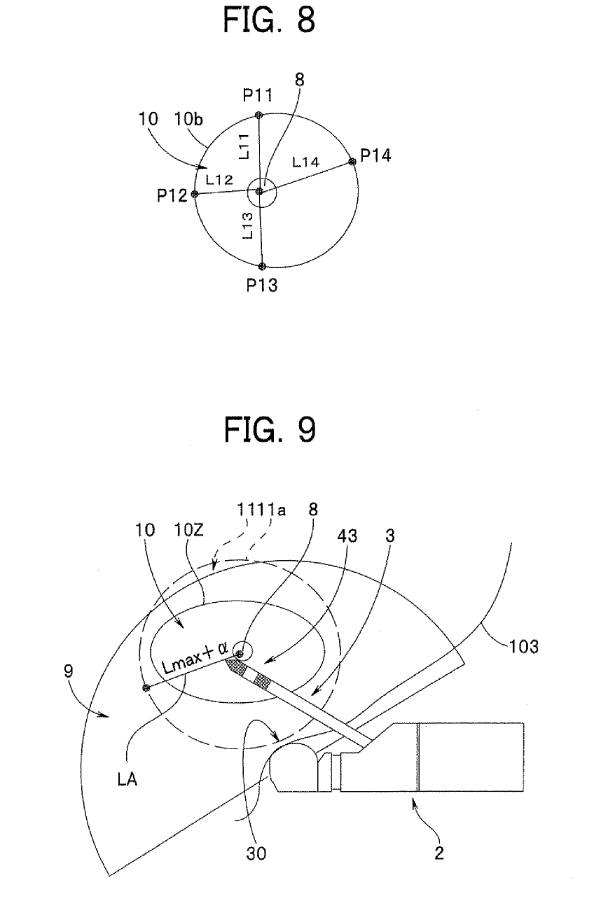

The operator then asks the staff to measure a distance from the marker 8 to an objective area peripheral border 10b as described above. Consequently, the staff selects, for example, four points P11, P12, P13, and P14 on the ultrasound image as shown in FIG. 8 and measures distances from the delineated marker 8. Reference numerals L11, L12, L13, and L14 denote the distances from the marker 8 as a base point to the points P11, 12, 13, and 14. Subsequently, if necessary, the operator moves the ultrasound transducer 30 to delineate an ultrasound image of a different cross section of the objective area 10 to measure the distances.

The operator determines a value Lmax+α, in which an arbitrary ablation margin α is added to a maximum value Lmax among a plurality of measurement results as shown in FIG. 9, as an ablation distance LA from the marker 8. Thus, the operator 101 sets a sphere 11, with the value Lmax+α as a radius, as the ablation target area and stores the value.

After setting the sphere 11 as an ablation target area, the operator arranges the electrode portion 43 of the ablation device 3 at a desired location in the objective area 10 and performs the ultrasound endoscope-guided ablation.

When the ablation of the objective area 10 has advanced for some degree, the operator instructs the measurement of an ablated part 10Z with the marker 8 as a base point. The operator checks whether the actual ablated part 10Z has reached a circumference 11a of the sphere 11 having preset Lmax+α as a radius.

The operator determines that the ablation is completed if the ablated area 10Z has reached the circumference 11a of the sphere 11 and if the ablated area 10Z has not reached the circumference 11a of the sphere 11 arranges the electrode portion 43 at the part to perform the ablation. The operator moves the ultrasound transducer 30 to check a plurality of locations.

In this way, a marker that is excellent in the ultrasound reflection intensity is placed in the objective area, and the distances from the marker as a base point to the objective area peripheral border are measured. A sphere of the ablation target area is set, with a value, in which a predetermined ablation margin is added to the maximum distance among the measurement results, as an ablation radius. Subsequently, the ablation device is arranged in the objective area to perform the ablation, and the distances from the marker after the ablation are measured. At this point, the objective area peripheral border on the ultrasound image becomes unclear due to the denaturation of the objective area and the denaturation of the surrounding tissues. However, the ablation of the objective area can be surely performed by checking whether the ablated part on the ultrasound image has reached Lmax+α based on the marker.

This solves a conventional problem, which has been occurring when the ablation target area is determined based on the objective area peripheral border, that with how much margin the ablation has been performed cannot be recognized as the delineated objective area peripheral border becomes unclear after the ablation. In other words, reliable ablation can be performed without depending on the skill of the operator and without leaving a diseased tissue.

As shown with a broken line in the ultrasound observation device 6, a storage unit 6m (see FIG. 6) may be arranged, and the ablation target area may be stored in the storage unit 6m. The configuration includes, for example, as shown in FIG. 9, a function of superimposing and displaying, on the ultrasound image and around the delineated marker 8, the circumference 11a of the sphere 11 with a radium Lmax+α shown with a broken line.

The ablation can be performed without losing the ablation target area by displaying the circumference 11a on the display device 7 when performing the ultrasound endoscope-guided ablation. Furthermore, whether the ablated area has reached Lmax+α can be easily and visually checked without measuring the ablated part.

The marker can also be checked on the ultrasound image after the ablation.

In general, the impedance and/or the temperature and/or the like are monitored in the ablation device during energization. Therefore, the output is terminated if the output exceeds a preset threshold, preventing the application of heat such that the tissues carbonize.

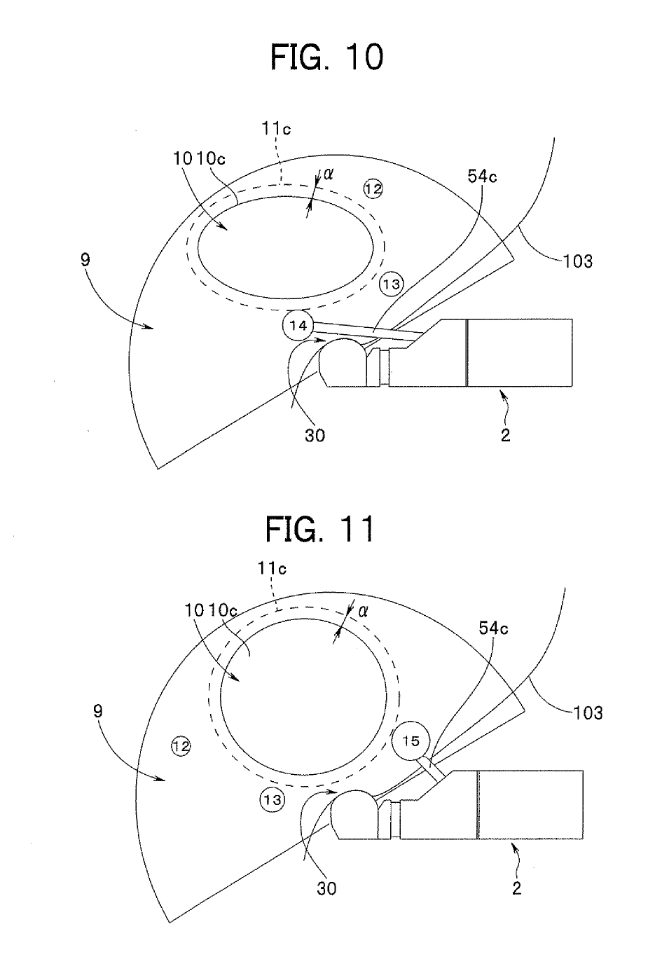

Another ablation method of the ultrasound endoscope-guided ablation system 1 will be described with reference to FIGS. 10 to 12. In the present embodiment, an injection needle, in which the pusher 55 and the marker 8 are excluded from the puncture needle 5, is used.

As in the above embodiment, the operator brings the ultrasound transducer 30 into contact with the lumen wall 103 and then causes the display device 7 to display the ultrasound image. The operator captures the objective area 10, such as cancer, at a desired location in the ultrasound scan area 9, as shown in FIG. 10.

The operator then uses the injection needle to place three markers 12, 13, and 14 at different locations that are outside and near the periphery of the objective area 10 under the guidance of the ultrasound endoscope. Thus, the operator punctures a needle tube 54c of the injection needle near the objective area 10 and injects, for example, jelly with contrast agent or with air bubbles.

At this point, the injection volume of the jelly is adjusted to change the sizes of the markers 12, 13, and 14. As a result, three markers 12, 13, and 14 in different sizes are placed in a scan plane.

After checking the placement of the markers 12, 13, and 14 near the objective area 10, the operator asks the staff to measure distances from the markers 12, 13, and 14 to the peripheral border of the objective area 10.

In this case, instead of measuring a plurality of points of the peripheral border, the staff, for example, traces the peripheral border of the objective area delineated on the display device 7 with an input pen described below to acquire a peripheral line and then computes and obtains, with a computing unit described below, an objective area peripheral line 10c based on the center of the delineated markers 12, 13, and 14. The staff also specifies an ablation margin α with an ablation margin setting unit described below. Consequently, an ablation target area setting line (hereinafter abbreviated as an ablation line) 11c, in which the ablation margin α is added to the objective area peripheral line 10c, shown with a broken line is superimposed and displayed on the display device 7.

The staff registers the absolute objective area peripheral line 10c and the ablation line 11c, with the delineated markers 12, 13, and 14 as base points, in the storage unit 6m.

The operator then performs, for example, a hand-side operation for moving the distal end side of the EUS 2 around an axis connecting the markers 12 and 13 to move the ultrasound transducer 30. Consequently, an ultrasound image of the objective area 10 of a different cross section, in which the markers 12 and 13 are displayed on the display device 7 as shown in FIG. 11, is delineated.

The operator uses the injection needle to place a marker 15 with a large diameter, the size of which is different from the markers 12, 13, and 14. As a result, the markers 12, 13, and 15 are placed in a scan plane. Therefore, four markers 12, 13, 14, and 15 are placed around the objective area 10 without being arranged within one plane.

As described above, the operator asks the staff to acquire an absolute objective area peripheral line 10d and an ablation line 11d with the delineated markers 12, 13, and 15 as base points and to register the lines in the storage unit 6m.

After acquiring and registering the objective area peripheral line 10d and the ablation line 11d, the operator and the staff move the ultrasound transducer 30 to acquire an absolute objective area peripheral border (not shown) and an ablation line (not shown) with the delineated markers 13, 14, and 15 as base points and to register the border and the line in the storage unit 6m. The operator and the staff then acquire an absolute objective area peripheral border (not shown) and an ablation line (not shown) with the delineated markers 12, 14, and 15 as base points and register the border and the line in the storage unit 6m.

With the series of operations, the acquisition of four absolute ablation lines with respect to the cross section including three markers among four markers 12, 13, 14, and 15 is completed.

In the above embodiment, the distal end side of the EUS 2 is moved around the axis connecting the markers 12 and 13 to obtain the ultrasound image, which is displayed with the markers 12 and 13, of a different cross section of the objective area 10 upon the placement of the fourth marker. However, the distal end side of the EUS 2 may be moved around the axis connecting the markers 12 and 14 to place the fourth marker, or the distal end side of the EUS 2 may be moved around the axis connecting the markers 13 and 14 to place the fourth marker.

In the present embodiment, the injection volume of the jelly is adjusted to allow identification of a plurality of markers. However, as shown in FIG. 48 described below, the lengths of the markers may be changed to allow the identification of the plurality of markers.

In the present embodiment, the ultrasound observation device 6 further includes a peripheral border input unit, a computing unit, and an ablation margin setting unit that are described below.

In the present embodiment, the staff extracts the peripheral line of the objective area. However, the peripheral line of the objective area may be automatically extracted by a computer installed in the ultrasound observation device 6.

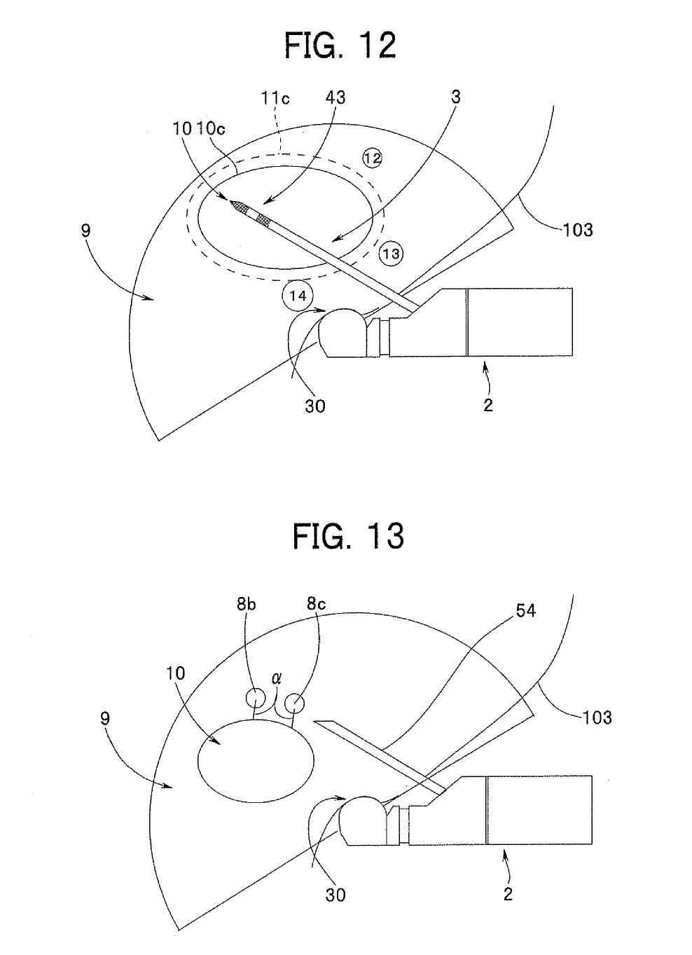

After completing the acquisition of the ablation lines 11c, 11d, . . . with respect to four cross sections, the operator arranges the electrode portion 43 of the ablation device 3 at a desired location in the objective area 10 as shown in FIG. 12 to perform the ultrasound endoscope-guided ablation.

The operator checks an actual ablation state when the ablation of the objective area 10 has advanced to some degree. More specifically, the operator checks whether the actual ablated area has reached the ablation line 11c on the plane including the markers 12, 13, and 14, checks whether the actual ablated area has reached the ablation line 11d on the plane including the markers 12, 13, and 15, checks whether the actual ablated area has reached the ablation line on the plane including the markers 13, 14, and 15, and checks whether the actual ablated area has reached the ablation line on the plane including the markers 12, 14, and 15, while performing the operation to move the ultrasound transducer 30 as necessary.

The operator determines that the ablation is completed if the ablated area has reached all ablation lines. On the other hand, the operator arranges the electrode portion 43 at a desired location on the ultrasound image to perform the ablation if the ablated area has not reached any of the ablation lines.

In this way, four markers are placed in predetermined states, and an ablation line is set for each cross section based on three of the four markers. The objective area is ablated while whether the ablated area has reached the ablation line of each surface is checked. This enables to obtain similar effects and advantages as in the above described embodiment.

The present embodiment enables to shorten the treatment time because the ablation target area can be reduced as compared to the case in which the circumference of the sphere of the ablation target area is set with Lmax+α.

Another ablation method of the ultrasound endoscope-guided ablation system 1 will be described with reference to FIGS. 13 to 16B.

The puncture needle 5 is used in the present embodiment. As in the embodiment described above, the operator brings the ultrasound transducer 30 into contact with the lumen wall 103 and then displays the ultrasound image on the display device 7. The operator then captures the objective area 10, such as cancer, at a desired location in the ultrasound scan area 9 as shown in FIG. 13.

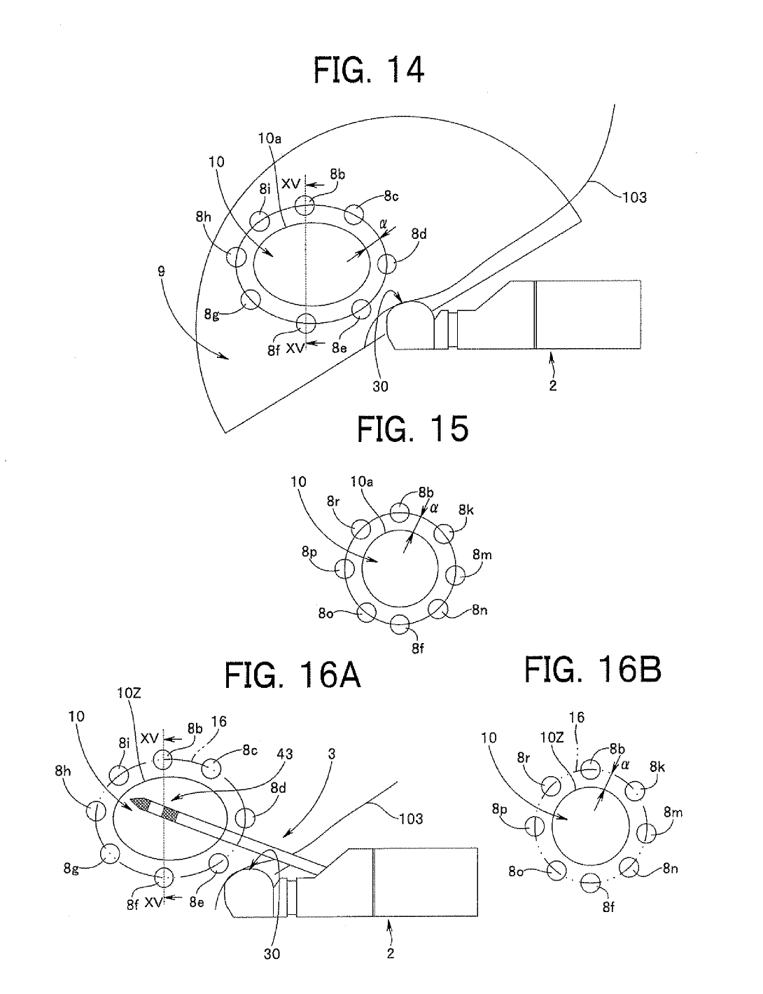

Under the guidance of the ultrasound endoscope, the operator sequentially places markers 8b, 8c, . . . at locations separated by a distance of the ablation margin α, in consideration of the ablation margin α from the objective area peripheral line 10a of the objective area 10. As a result, markers 8b, 8c, . . . , and 8i are placed, at locations with the ablation margin α from the objective area peripheral line 10a, within a single scan plane as shown in FIG. 14.

The operator then moves the ultrasound transducer 30 to display an ultrasound image of a XV-XV cross section of FIG. 14 on the display device 7. Consequently, the display device 7 displays the markers 8b, 8f, and the objective area 10, as shown in FIG. 15. At this point, under the guidance of the ultrasound endoscope, the operator sequentially places markers 8k, 8m, . . . , 8p, and 8r at locations with the ablation margin α from the objective area peripheral line 10a of the objective area 10.

After placing the plurality of markers 8b, . . . , and 8r, the operator arranges the electrode portion 43 of the ablation device 3 at a desired location in the objective area 10 as shown in FIG. 16A and performs the ultrasound endoscope-guided ablation.

When the ablation of the objective area 10 has advanced to some degree, the operator checks whether the actual ablated part 10Z has reached an ablation line defined by a virtual line 16 connecting the markers shown in FIGS. 16A and 16B.

The operator determines that the ablation is completed if the operator can check that the ablated area has reached the virtual line connecting the markers. On the other hand, the operator arranges the electrode portion 43 to perform the ablation if the actual ablated area has not reached the virtual line.

In this way, the arrangement of the plurality of markers around the objective area in advance, taking the ablation margin α into consideration, allows the operator to check whether the ablated area has reached the virtual line connecting the makers to thereby surely perform the ablation of the objective area.

In the above described embodiment, an efficient procedure can be achieved by using a two-dimensional array as an ultrasound transducer or installing a two-plane scan type ultrasound transducer to delineate a plurality of ultrasound images of different scanned surfaces at the same time.

Alternatively, an extracorporeal probe may be brought into contact with the body surface to capture the objective area that is captured by the ultrasound transducer 30. In this configuration, the same objective area 10, the marker 8, the needle tube 54 of the marker placing device 5, and the electrode portion 43 of the ablation device 3 are displayed in the ultrasound image of the EUS 2 and the ultrasound image of the extracorporeal probe.

This enables to easily figure out the entire objective area 10 and to easily perform the orientation to the objective area 10. Therefore, a procedure can be more quickly and surely performed, allowing to shorten the procedure time and to reduce the burden of the operator.

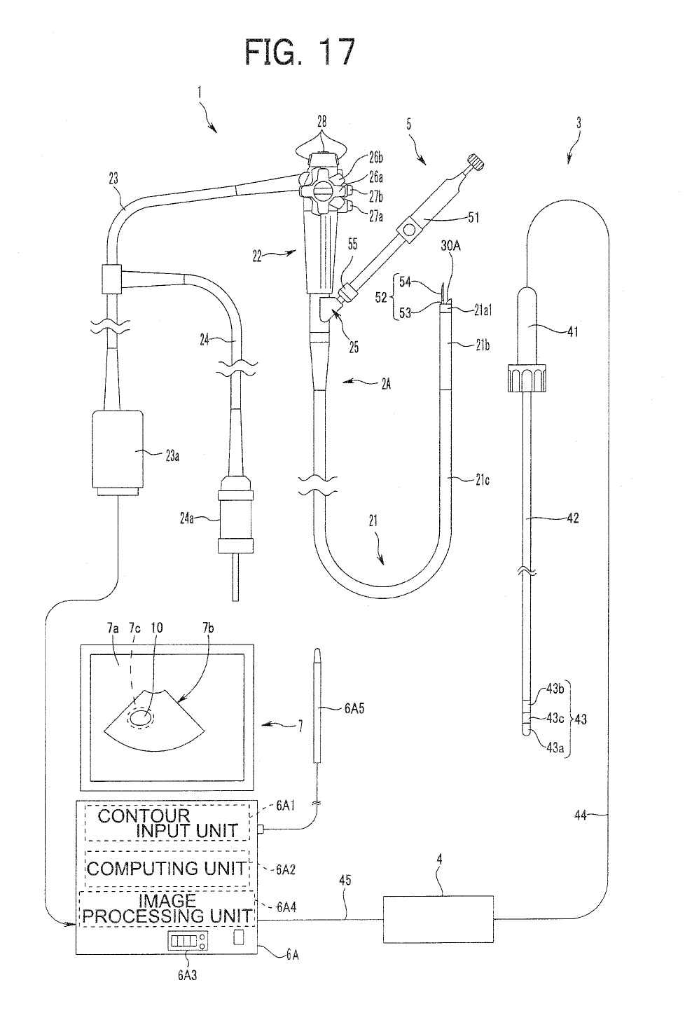

A second embodiment of the present invention will be described with reference to FIGS. 17 to 37.

As shown in FIG. 17, configurations of an EUS 2A and an ultrasound observation device 6A of the first embodiment are different in an ultrasound endoscope-guided ablation system 1A of the present embodiment.

The ultrasound observation device 6A includes, as an ablation target area specifying unit, a peripheral border input unit 6A1, a computing unit 6A2, an ablation margin setting unit 6A3, and an image processing unit 6A4. Reference numeral 6A5 denotes an input pen.

The input pen 6A5 is an objective area input function unit and is a device for tracing a line of the peripheral border of the objective area 10 on an ultrasound image 7b displayed on a screen 7a to acquire information of the objective area peripheral line. The peripheral line traced by the input pen 6A5 is displayed on the screen 7a through an image processing unit 6A4 described below. Information of the displayed peripheral line and the like is outputted to a peripheral border input unit 6A1 from the input pen 6A5.

The computing unit 6A2 extracts objective area peripheral line data from the information of the peripheral line inputted to the peripheral border input unit 6A and stores the data in the storage unit 6m.

The ablation margin setting unit 6A3 is a margin input function unit that sets an ablation margin necessary for the operator to manually ablate the objective area 10. Once the operator sets the ablation margin α to the ablation margin setting unit 6A3, the computing unit 6A2 extracts ablation line data, which is ablation target area data in which the ablation margin α is added to the objective area peripheral line data, and stores the data in the storage unit 6m.

The image processing unit 6A4 includes, in addition to a normal image processing function of an ultrasound observation device, a function to apply a predetermined process to the objective area peripheral line data and the ablation line data extracted by the computing unit 6A2 to superimpose and display the ultrasound image 7b as well as an ablation line 7c, as shown with a broken line as an example, on the screen 7a.

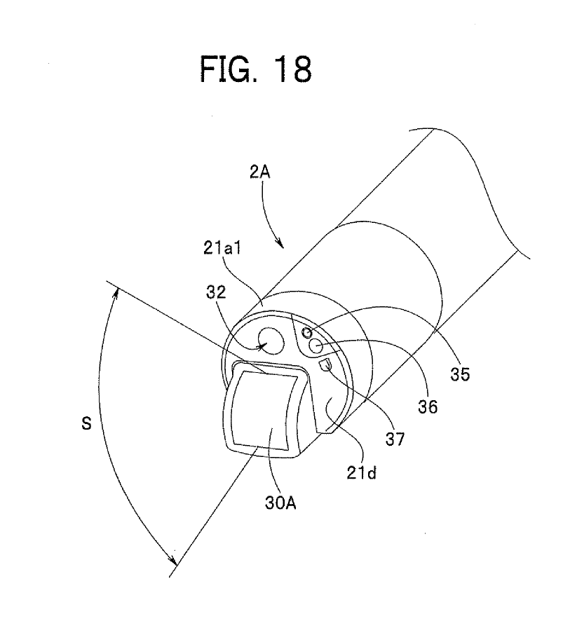

As shown in FIG. 18, in the EUS 2A, a distal end surface 21d of a distal end rigid portion 21a1 constituting the insertion portion 21 is constituted by a plane which is orthogonal to the longitudinal axis direction. An electronically scanning ultrasound transducer 30A protrudes from the distal end surface, and as in the first embodiment, is configured as a convex array in which a plurality of ultrasound elements are aligned.

As in the first embodiment, the distal end opening 32, the illumination lens cover 35, the observation lens cover 36, and the air and water feeding nozzle 37 are arranged on the distal end surface 21d. Thus, the observation optical system of the EUS 2 is an oblique-view type, while the observation optical system of the EUS 2A is a forward-view type.

A forceps raising table is swingably arranged near the distal end opening 32. Other configurations are similar to the first embodiment, and the same members are designated with the same reference numerals and the description will not be repeated. Although the ultrasound endoscope is EUS 2A in the present embodiment, but the EUS 2 could be used as the ultrasound endoscope.

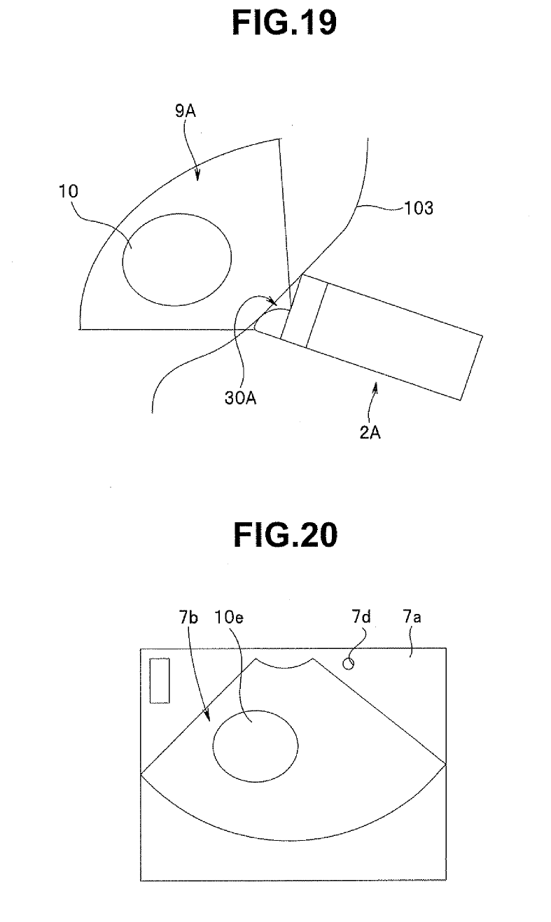

An ablation method of the ultrasound endoscope-guided ablation system 1A configured as described above will be described with reference to FIGS. 19 to 24.

As in the above described embodiment, the operator brings the ultrasound transducer 30A into contact with the lumen wall 103 as shown in FIG. 19. The operator captures the objective area 10, such as cancer, at a desired location in an ultrasound scan area 9A. Consequently, as shown in FIG. 20, a delineated image 10e of the objective area 10 is delineated on the ultrasound image 7b of the screen 7a of the display device 7. Reference numeral 7d denotes a projection indication that indicates a delivery start location of a treatment instrument such as a puncture needle.

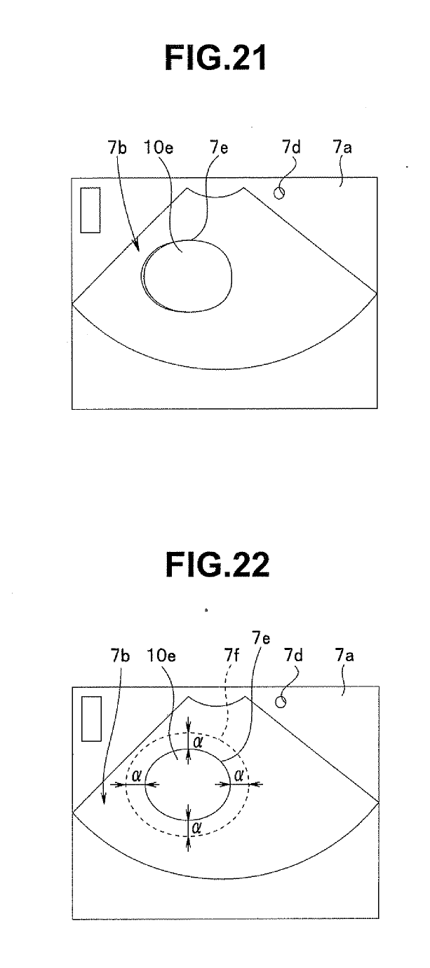

When the delineated image 10e of the objective area 10 is delineated at the desired location on the ultrasound image 7b, the operator instructs the staff to acquire the peripheral border of the objective area 10. The staff traces the peripheral border of the delineated image 10e displayed on the display device 7 with the input pen 6A5 and delineates a peripheral line 7e of the delineated image 10e on the screen 7a as shown in FIG. 21. Information of the peripheral line 7e delineated on the screen 7a is outputted to the peripheral border input unit 6A1, extracted by the computing unit 6A2 as objective area peripheral line data, and stored in the storage unit 6m.

While instructing the acquisition of the peripheral border, the operator observes the delineated image 10e and the situation in the surrounding to determine the ablation margin. Subsequently, the operator or the staff inputs the determined ablation margin α in the ablation margin setting unit 6A3.

Once the ablation margin α is inputted to the ablation margin setting unit 6A3, the computing unit 6A2 calculates ablation line data indicative of an ablation target area in which the ablation margin α is added to the extracted objective area peripheral line data. The calculated ablation line data is registered in the storage unit 6m and outputted to the image processing unit 6A4. The image processing unit 6A4 applies a predetermined process to the ablation line data and outputs the data to the display device 7. Consequently, an ablation line 7f is superimposed on the delineated image 10e and displayed on the screen 7a as shown with a broken line of FIG. 22.

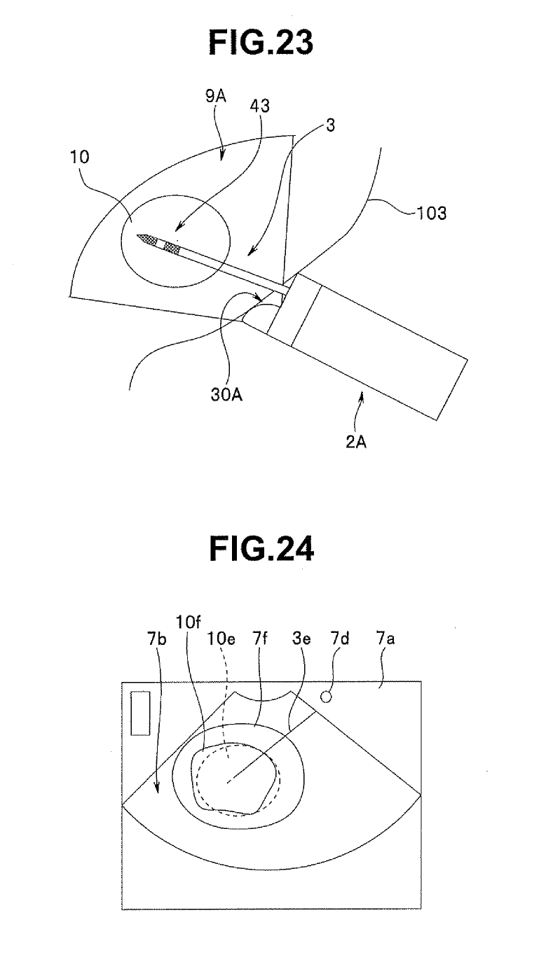

When the ablation line 7f shown in FIG. 22 is displayed on the ultrasound image 7b, the operator holds the ultrasound transducer 30A of the EUS 2A such that the ultrasound transducer 30A does not move and arranges the electrode portion 43 of the ablation device 3 at a desired location in the objective area 10 as shown in FIG. 23.

The ultrasound endoscope-guided ablation is then started. Consequently, as the ablation progresses, the delineated image 10e displayed on the ultrasound image 7b gradually becomes unclear as shown in FIG. 24. Meanwhile, an ablated objective part ablation image 10f is displayed in place of the delineated image 10e. Reference numeral 3e denotes a delineated image of the ablation device 3 including the electrode portion 43.

The operator performs a hand-side operation of only the ablation device 3 to continue the ablation such that the objective part ablation image 10f reaches the ablation line 7f. The operator then checks that the objective part ablation image 10f displayed on the ultrasound image 7b has reached the ablation line 7f and ends the ablation of the objective area 10.

In this way, the operator traces the objective area displayed on the ultrasound image with an input pen to acquire objective area peripheral line data. In addition, the operator inputs an ablation margin to the ablation margin setting unit to obtain ablation line data, in which the ablation margin α is added to the objective area peripheral line data, and displays the ablation line in the ultrasound image. The operator ablates the objective area with the ablation line being displayed and performs the ablation while checking whether the objective part ablation image has reached the ablation line. This enables to obtain similar effects and advantages as in the above described embodiment.

In the present embodiment, the objective area is traced by the input pen to acquire the objective area peripheral line data. However, the operator or the staff may operate a pointing device such as a mouse or a trackball to instruct a rough area of the objective area, and based on the instructed information, the ultrasound observation device may automatically extract the boundary between the objective area and the surrounding tissues.

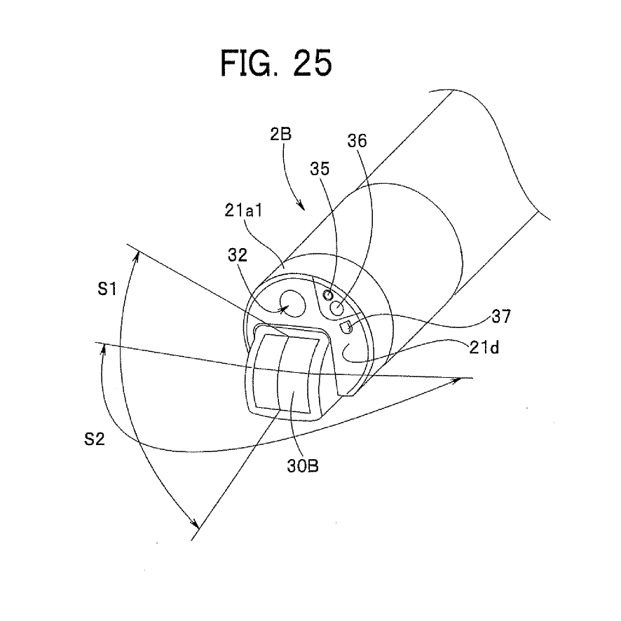

Another ablation method of the ultrasound endoscope-guided ablation system 1A will be described with reference to FIGS. 25 to 31. An EUS 2B shown in FIG. 25 is used in place of the EUS 2A in the present embodiment.

The EUS 2B shown in FIG. 25 includes an ultrasound transducer 30B. The ultrasound transducer 30B uses a two-dimensional array type or the like to be controlled by the ultrasound observation device 6A to scan two orthogonal surfaces to thereby obtain a first scanned surface S1 and a second scanned surface S2. Other configurations are similar to the above described embodiment, and the same members are designated with the same reference numerals and the description will not be repeated.

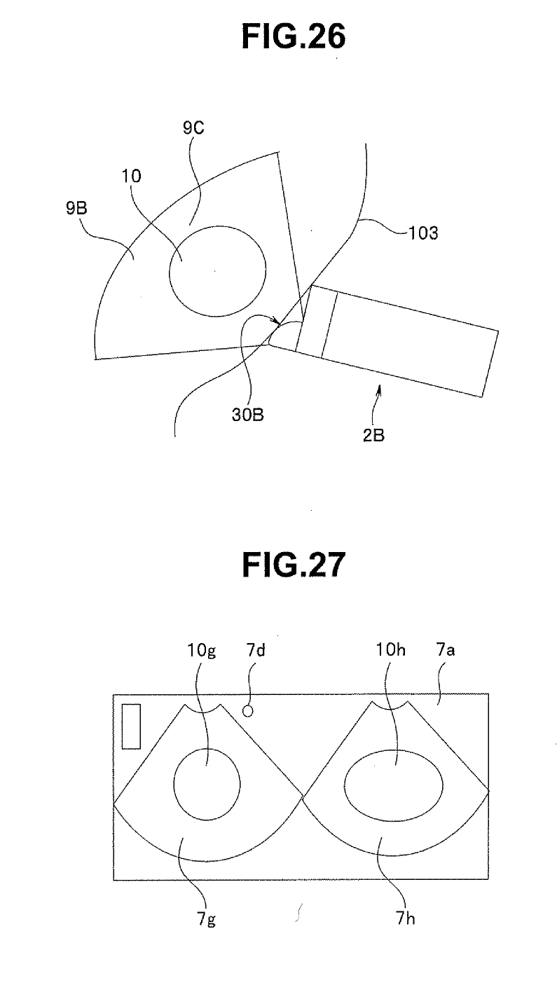

In the EUS 2B of the present embodiment, the operator brings the ultrasound transducer 30B into contact with the lumen wall 103, and as shown in FIG. 26, captures the objective area 10, such as cancer, at a desired location in an ultrasound scan area 9B of the first scan surface S1 and in an ultrasound scan area 9C of the second scanned surface S2.

Consequently, as shown in FIG. 27, a first ultrasound image 7g including a delineated image 10g captured in the first ultrasound scan area 9B and a second ultrasound image 7h including a delineated image 10h captured in the second ultrasound scan area 9C are displayed on the screen 7a. When the delineated images 10g and 10h are displayed in desired states on two ultrasound images 7g and 7h displayed on the screen 7a, the operator instructs the staff to acquire the peripheral border of the objective area 10.

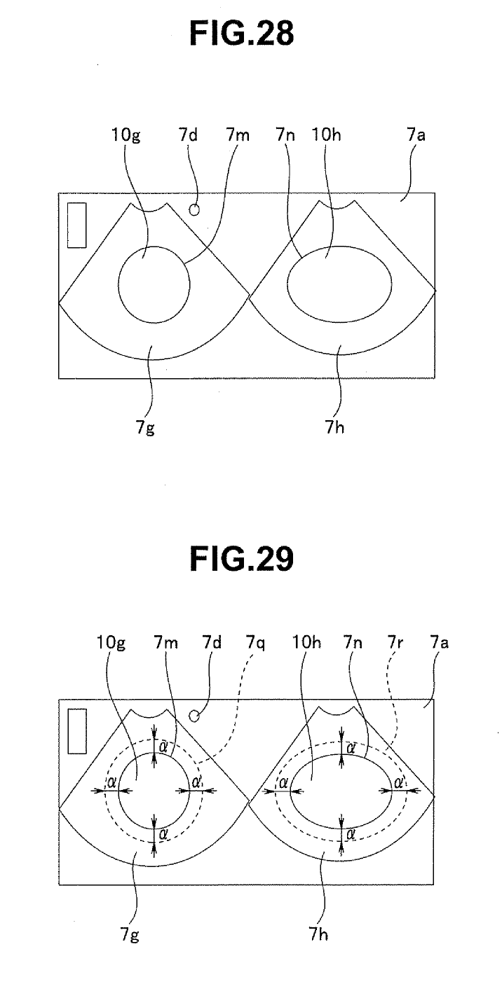

In the present embodiment, the staff traces the peripheral borders of the delineated images 10g and 10h displayed on the display device 7 with the input pen 6A5 to delineate peripheral lines 7m and 7n of the delineated images 10g and 10h on the screen 7a as shown in FIG. 28. Information of the peripheral lines 7m and 7n delineated on the screen 7a is outputted to the peripheral border input unit 6A1, extracted by the computing unit 6A2 as objective area peripheral line data of the peripheral lines 7m and 7n, and stored in the storage unit 6m.

The operator instructs the acquisition of the peripheral border while observing the situations in the delineated images 10g, 10h, and the surrounding to determine the ablation margin. Subsequently, the operator or the staff inputs the determined ablation margin α in the ablation margin setting unit 6A3.

Once the ablation margin α is inputted to the ablation margin setting unit 6A3, the computing unit 6A2 calculates each of the ablation line data, in which the ablation margin α is added to each of the extracted objective area peripheral line data. Each of the calculated ablation line data is registered in the storage unit 6m and outputted to the image processing unit 6A4. The image processing unit 6A4 applies a predetermined process to each of the ablation line data and outputs the data to the display device 7. Consequently, ablation lines 7q and 7r are superimposed on the delineated image 10g and displayed on the screen 7a as shown with a broken line of FIG. 29.

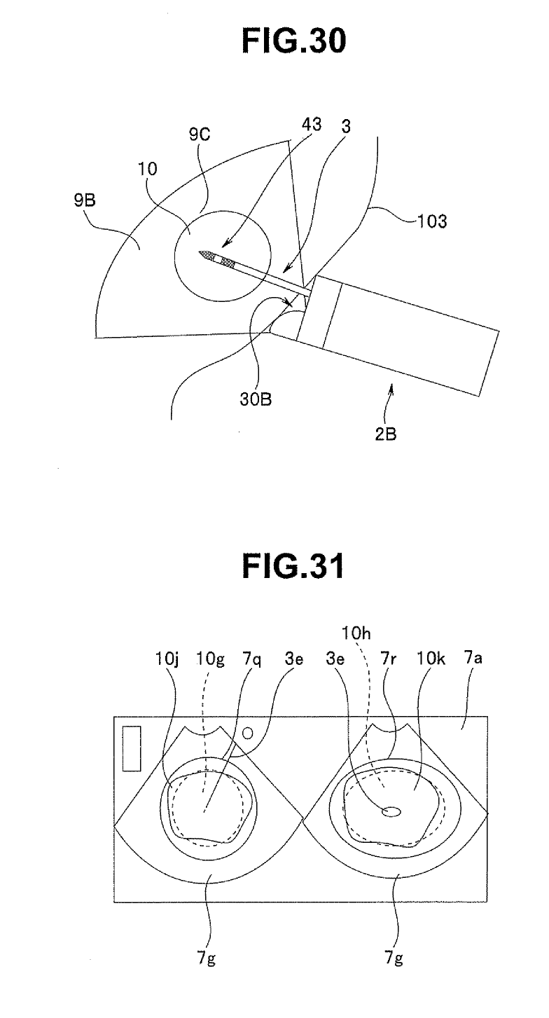

When the ablation lines 7q and 7r shown in FIG. 29 are displayed on the ultrasound image 7b, the operator holds the ultrasound transducer 30B of the EUS 2B such that the ultrasound transducer 30B does not move and arranges the electrode portion 43 of the ablation device 3 at a desired location in the objective area 10 as shown in FIG. 30.

The ultrasound endoscope-guided ablation is then started. Consequently, as the ablation progresses, the delineated images 10g and 10h displayed in the ultrasound images 7g and 7h become gradually unclear as shown in FIG. 31, and meanwhile, ablated objective part ablation images 10j and 10k are displayed in place of the delineated images 10g and 10h.

The operator performs a hand-side operation of only the ablation device 3 to continue the ablation such that the objective part ablation images 10j and 10k displayed in the ultrasound images 7g and 7h reach the ablation lines 7q and 7r, respectively.

The operator checks that the objective part ablation image 10j displayed in the ultrasound image 7g has reached the ablation line 7q and that the objective part ablation image 10k displayed in the ultrasound image 7h has reached the ablation line 7r and ends the ablation of the objective area 10.

In this way, the two-dimensional array type ultrasound transducer is driven so as to scan two orthogonal planes to thereby display two ultrasound images on the screen. The objective area is displayed on each ultrasound image to acquire the objective area peripheral line data as well as to acquire the ablation line data in which the ablation margin α is added to the objective area peripheral line data. More accurate management of the ablation margin is made possible by displaying the ablation line in each ultrasound image during the procedure of the ablation to check whether the objective part ablation image has reached the ablation line.

Managing the ablation of the objective area with two planes enables to deal with a flat objective area, a complex objective area, and the like.

Although the two planes are orthogonal in the present embodiment, the angle could be set arbitrarily. Other effects and advantages are similar as in the above described embodiment.

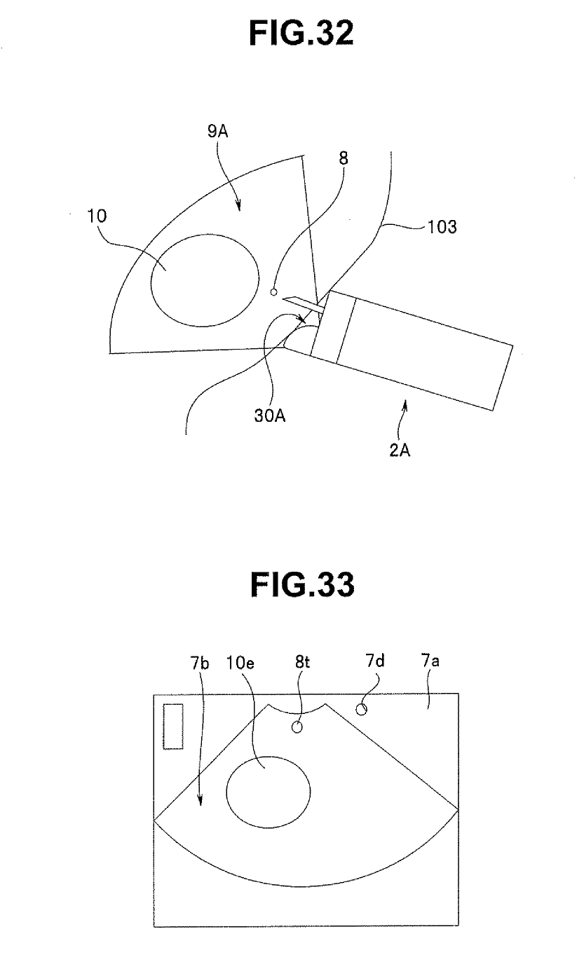

Another ablation method of the ultrasound endoscope-guided ablation system 1A will be described with reference to FIGS. 32 to 37. The puncture needle 5 as well as the EUS 2A shown in FIGS. 19 to 24 are used in the present embodiment. However, the EUS is not limited to the EUS 2A, but may also be the EUS 2B.

As in the above described embodiment, the operator brings the ultrasound transducer 30A into contact with the lumen wall 103 as shown in FIG. 32. The operator then captures the objective area 10, such as cancer, to a desired location in the ultrasound scan area 9A. Consequently, the delineated image 10e of the objective area 10 is delineated on the ultrasound image 7b of the screen 7a of the display device 7 as shown in FIG. 33.

The operator 101 then arranges the distal end of the needle tube 54 near the objective area 10 under the guidance of the ultrasound endoscope to place the marker 8 with the above described procedure. Consequently, a marker image 8t is delineated on the ultrasound image 7b of FIG. 33.

After checking the placement of the marker 8 to the objective area 10, the operator 101 instructs the staff to acquire the peripheral border of the objective area 10.

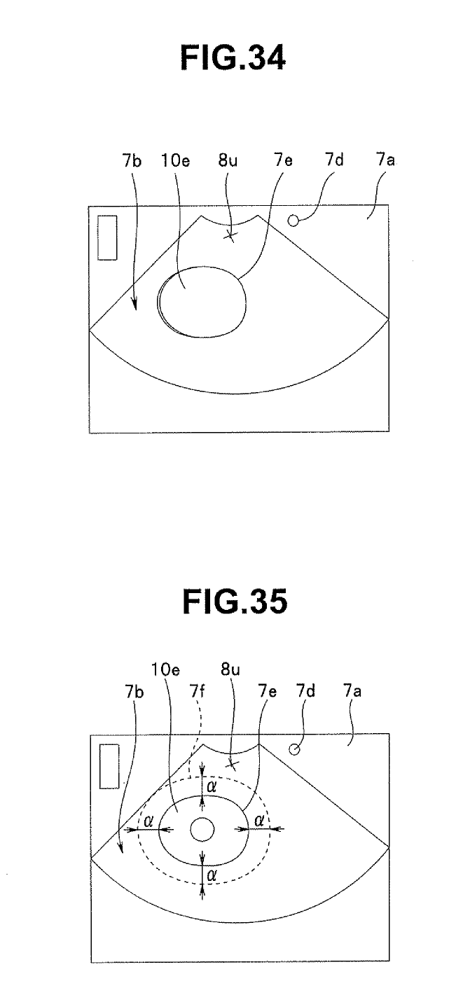

At this point, the staff traces the peripheral border of the delineated image 10e displayed on the display device 7 with the input pen 6A5 as described above and specifies the marker image 8t. Consequently, the peripheral line 7e of the delineated image 10e and an indicator 8u indicative of the location of the marker image 8t are displayed on the screen 7a as shown in FIG. 34. Information of the locations of the peripheral line 7e and the indicator 8u are outputted to the peripheral border input unit 6A1, and the computing unit 6A2 stores the information in the storage unit 6m as objective area peripheral line data with the location of the indicator 8u as a base point.

As described above, the operator 101 or the staff inputs the ablation margin a in the ablation margin setting unit 6A3 and acquires the ablation line 7f superimposed on the delineated image 10e and shown with a broken line as shown in FIG. 35.

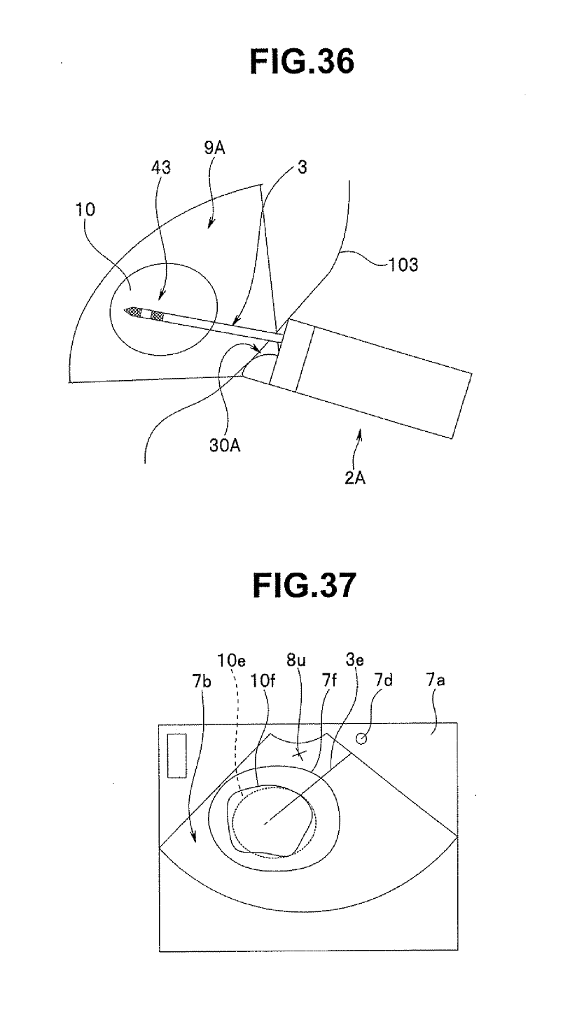

Subsequently, as in the above described embodiment, the electrode portion 43 of the ablation device 3 is arranged in the objective area 10 as shown in FIG. 36 to perform the ultrasound endoscope-guided ablation.

The operator 101 ablates the objective area 10 while checking whether the objective part ablation image 10f displayed on the ultrasound image 7b as shown in FIG. 37 has reached the ablation line 7f. The operator 101 then checks that the objective part ablation image 10f has reached the ablation line 7f and ends the ablation of the objective area 10.

In the present embodiment, the operator 101 or the staff always checks the location of the marker image 8t during the procedure. Therefore, checking the displacement of the marker image 8t based on the indicator 8u indicative of the location of the marker image 8t enables to easily determine that the ultrasound transducer 30A has moved. In response, if, by any chance, the ultrasound transducer 30A has moved, a hand-side operation is performed to match the indicator 8u with the marker image 8t. As a result, the ablation target area that has once been displaced is reset to the original ablation target area.

In this way, the location of the marker image is also acquired upon the acquisition of the objective area peripheral line data of the objective area displayed on the ultrasound image. Specifically, the objective area peripheral line of the objective area is acquired based on the marker. This enables to easily determine that the ultrasound transducer has moved during the procedure and to easily put back the location of the ultrasound transducer to the original state to perform the ablation even if the ultrasound transducer has moved during the procedure. Other effects and advantages are similar as in the above described embodiment.

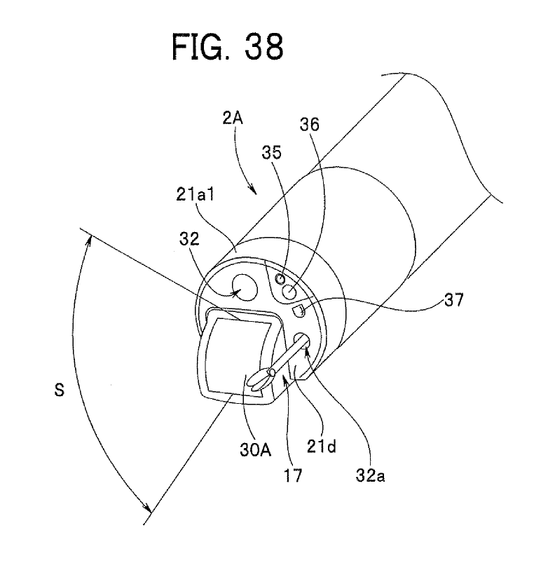

A second distal end opening 32a that is in communication with a second treatment instrument channel may be arranged on the distal end surface 21d of the EUS 2A as shown in FIG. 38 as an example to prevent the movement of the ultrasound transducer during the procedure.

In this configuration, grasping forceps 17 are projected from the second distal end opening 32a, and the grasping forceps 17 grasp a body tissue near the ultrasound transducer 30A. This enables to prevent the movement of the ultrasound transducer 30A and perform the ablation.

The ablation device 3 is also used endoscopically. Therefore, the outer dimension or the hardness length of the ablation device 3 is limited. Thus, the ablation performance of the ablation device 3 tends to be poorer than that in an ablation device used percutaneously. Therefore, it is difficult to ablate the entire objective area of a large lesion or the like at once, and more than one ablation has been performed on the objective area. Thus, the procedure has been long.

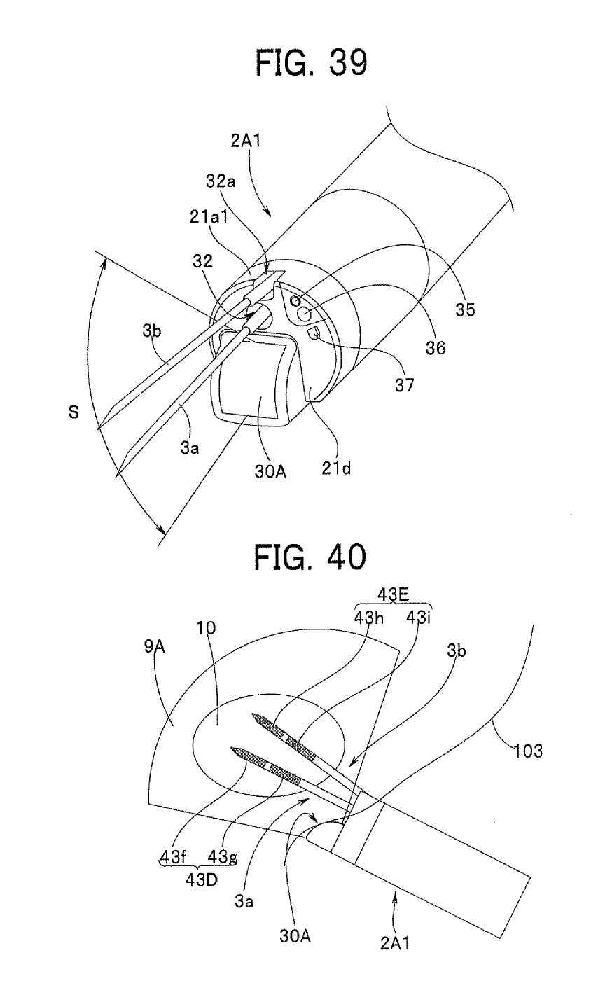

To solve the problem, for example, the distal end surface 21d of an EUS 2A1 shown in FIG. 39 includes the second distal end opening 32a in communication with the second treatment instrument channel. As a result, two ablation devices 3a and 3b are projected in the EUS 2A1, thereby enabling to arrange electrode portions 43D and 43E in the objective area 10 as shown in FIG. 40.

In this configuration, a first ablation device 3a is projected from the first distal end opening 32, and as shown in FIG. 40, an electrode portion 43D is arranged not at the center of the objective area, but is displaced in the lower peripheral border direction of FIG. 40. Subsequently, a second ablation device 3b is projected from the second distal end opening 32a, and an electrode portion 43E is arranged in the upper peripheral border direction of FIG. 40 in the objective area 10, apart from the electrode portion 43D.

Radio frequencies are simultaneously outputted from the electrode portions 43D and 43E of two ablation devices 3a and 3b to perform the ablation. In the present embodiment, the electrode portions 43D and 43E include four electrodes 43f, 43g, 43h, and 43i in total, and therefore, the radio frequencies are outputted with all combinations of the electrodes. This allows the ablation on a wide scale at a time.

Arranging two ablation devices in the objective area and simultaneously outputting the radio frequencies from two electrode portions allow the ablation of a wide objective area in a short time at a time. Therefore, the procedure time can be shortened, and burdens of the patient and the operator can be reduced.

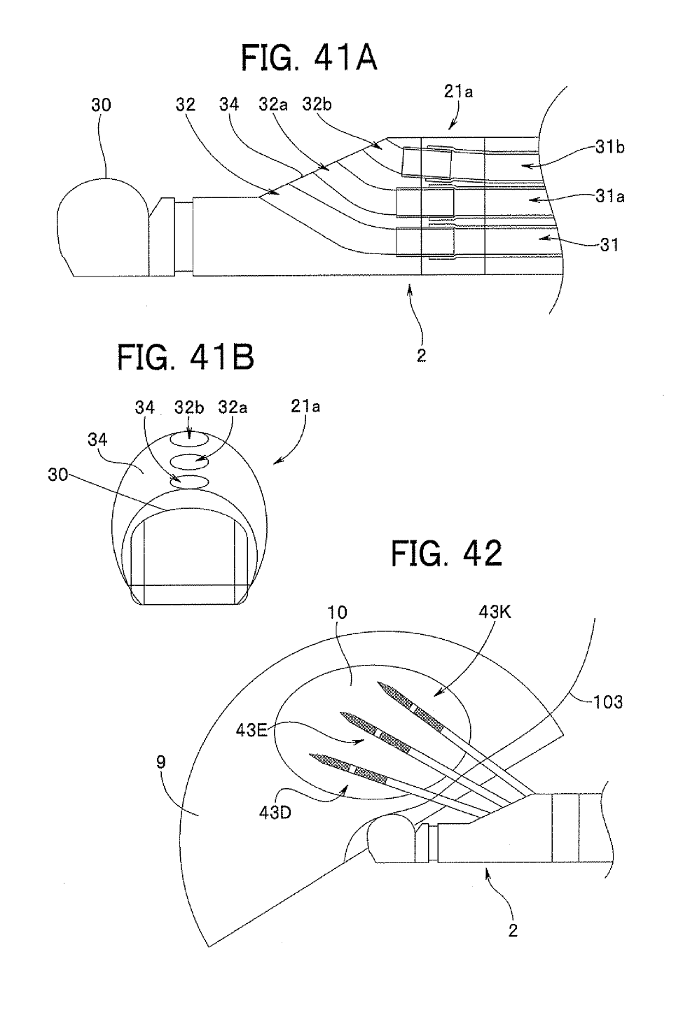

The number of the ablation devices punctured into the objective area is not limited to one or two, but may be more. For example, three distal end openings 32, 32a, and 32b may be arranged on the slope portion 34 of the distal end rigid portion 21a of the EUS 2 as shown in FIGS. 41A and 41B.

As a result, three electrode portions 43D, 43E, and 43K of the ablation devices 3a, 3b, and 3c are arranged in the objective area 10 through three treatment instrument channels as shown in FIG. 42, allowing the ablation of a still wider objective area 10 at a time. Reference numerals 31, 31a, and 31b denote treatment instrument channels.

Conventionally, the operator has reached the objective area with the following procedure when treating the objective area, such as cancer, with an EUS mounted with a convex ultrasound transducer.

The operator first delineates, on the display device, an organ, such as aorta, that can be most easily delineated and that is easy to be recognized in the ultrasound image and sets the organ as a base point. Subsequently, the operator moves the ultrasound transducer in the direction where the objective area anatomically exists. In this case, the operator sequentially delineates blood vessel, organ, and the like that serve as targets around the aorta to reach the objective area.

In the procedure, which part is being delineated cannot be determined in some cases when delineating a target in the middle. In that case, the operator had to return to the base point and start over from the first procedure.

The operator reaches the objective area 10 with the following procedure when treating the objective area 10 with the EUSs 2, 2A, and 2B mounted with the convex ultrasound transducers 30, 30A, and 30B.

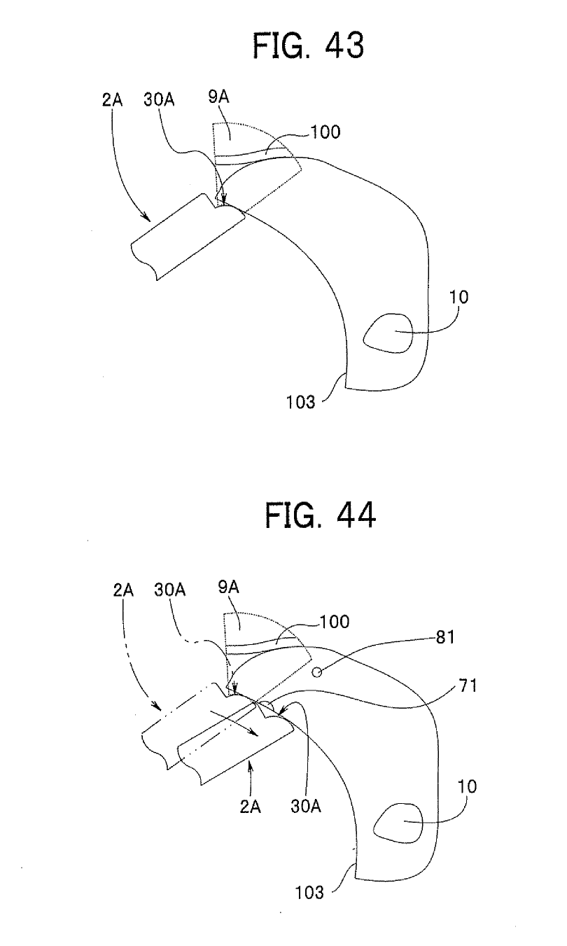

The operator first brings the ultrasound transducer 30A of the EUS 2A into contact with the lumen wall 103 as shown in FIG. 43, captures a large vessel 100, which is an organ serving as a base point such as aorta, in the ultrasound scan area 9A on the display device 7, and delineates the large vessel 100 on the ultrasound image.

The operator then performs a hand-side operation for moving the ultrasound transducer 30A from the base point into the arrow direction of FIG. 44 where the objective area anatomically exists. The operator then arranges the ultrasound transducer 30A at a location in the ultrasound image where the organ, such as celiac artery or lymph node, is delineated, the organ being an indication located on the way to the objective area.

The operator places a first endoscopic marker 71 on the lumen inner wall as shown in FIG. 44 to record the location when delineating the organ serving as a mark. The first endoscopic marker 71 is a marker designed to be seen on an endoscopic image. The operator places, as necessary, a first ultrasound marker 81, which is the marker 8, in the delineated organ or near the organ under the guidance of the ultrasound endoscope. The first ultrasound marker 81 is a marker designed to be delineated on the ultrasound image.

The placement of the endoscopic marker is performed by an injection needle not shown through a treatment instrument channel included in the EUS 2A. Meanwhile, the placement of the ultrasound marker is performed by the puncture needle 5 through the treatment instrument channel included in the EUS 2A. Alternatively, the ultrasound marker may be placed first by the puncture needle 5, and then the endoscopic marker may be placed using the same puncture needle 5. In this case, the placement of the endoscopic marker is performed by pulling out the pusher 55 of the puncture needle 5 and connecting a syringe full of pigment or stain solution to the ferrule portion 60. When using a pigment such as Indian ink, methylene blue, and indigo carmine, the puncture needle 5 is used to inject the pigment under mucosa to perform the placement. When using a stain solution such as iodine and methylene blue, the puncture needle 5 is used to disperse the pigment of the mucosal surface to stain the mucosa.

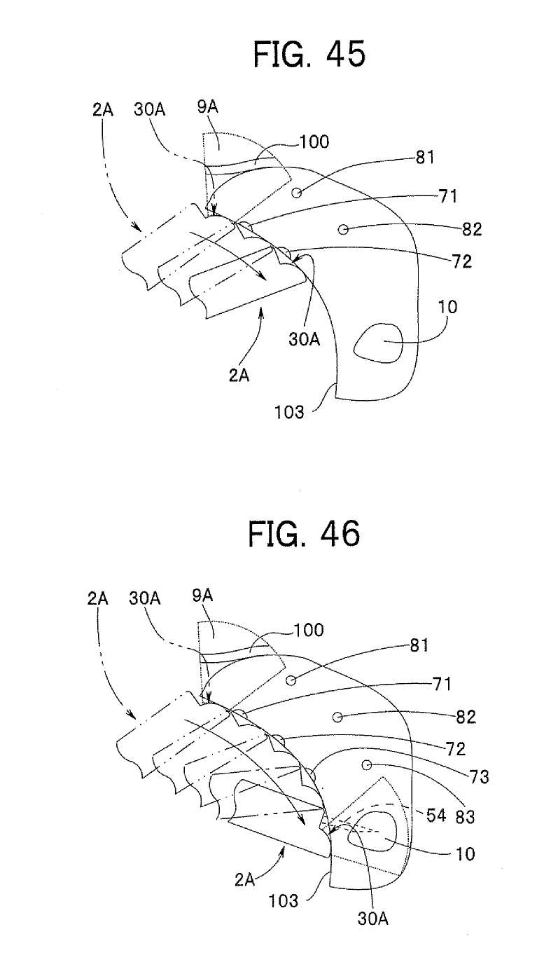

Subsequently, the operator continues a hand-side operation for moving the ultrasound transducer 30A in the arrow direction where the objective area anatomically exists as shown in FIGS. 45 and 46. The operator then arranges the ultrasound transducer 30A at a location where blood vessel, lymph node, or the like is located on the way to the objective area. The operator then places a second endoscopic marker 72 and a second ultrasound marker 82, a third endoscopic marker 73 and a third ultrasound marker 83, . . . every time the organ serving as a mark is delineated and records the trajectory of the movement of the ultrasound transducer 30A.

After placing, for example, the markers 73 and 83 as shown in FIG. 46, the objective area 10 can be captured in the ultrasound scan area 9A. The operator then performs the ultrasound observation of the objective area 10, puncture of the needle tube 54 as shown with a broken line, or other treatments.

In the present embodiment, if the operator, by any chance, loses the delineated location as described above while moving the ultrasound transducer 30A, the operator looks for the marker placed before the location. Thus, for example, if the operator loses the movement direction after placing the markers 73 and 84, the operator finds the placed markers 73 and 83 so that the delineation of organ can be restarted from the place.

The endoscopic marker is not limited to Indian ink, methylene blue, indigo carmine, or the like that can be easily checked in the endoscopic image. A material, such as a clip, that can be discriminated in the endoscopic image and that does not easily drop out can be utilized.

As described, in a series of movement operations from capturing the base point to reaching the objective area, the endoscopic marker and the ultrasound marker are placed every time an intermediate mark is captured. Consequently, in the process of moving the ultrasound transducer toward the objective area, even if the operator loses the orientation, the operator can return to the endoscopic marker and the ultrasound marker placed previously to restart the movement of the ultrasound transducer toward the objective area without restarting from the base point.

This can surely prevent the significantly extended procedure time even if the orientation is lost during the movement of the ultrasound transducer.

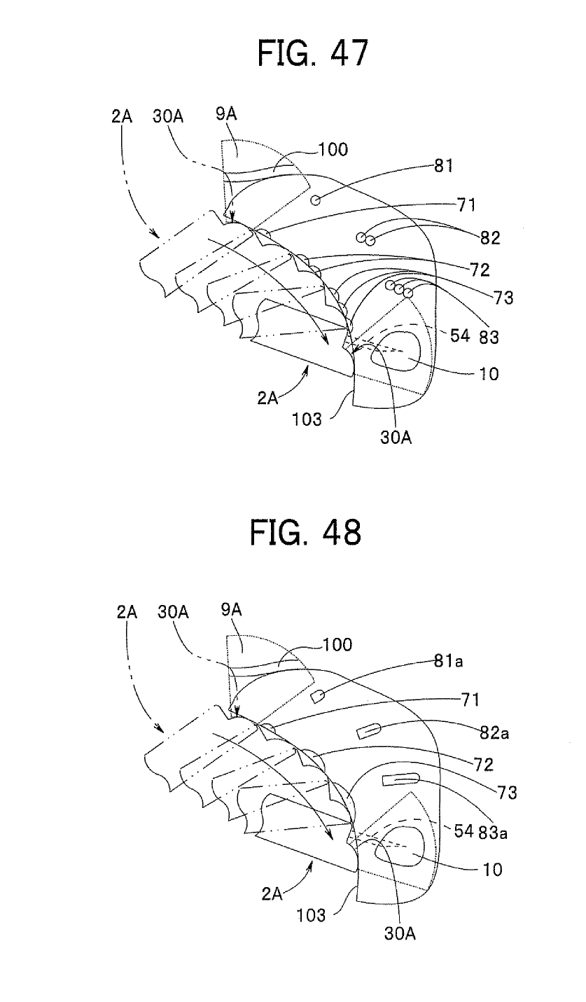

In the above described embodiment, it is difficult to immediately determine, at a glance of a plurality of markers, the order of the placement of the markers from the base point. Therefore, the number of the endoscopic markers 71, 72, and 73 and the number of the ultrasound markers 81, 82, and 83 are increased as shown in FIG. 47 every time the ultrasound transducer 30A is moved to enable to clearly determine the order of movement of the ultrasound transducer 30A. Alternatively, the sizes of the endoscopic markers 71, 72, and 73 and the sizes of the ultrasound markers 81, 82, and 83 may be increased as shown in FIG. 48 every time the ultrasound transducer 30A is moved.

In the endoscopic markers 71, 72, and 73 of FIG. 48, the injection volume of the pigment is increased upon every movement. Meanwhile, in the ultrasound markers 81a, 82a, and 83a, the lengths are increased by a predetermined length upon every movement.

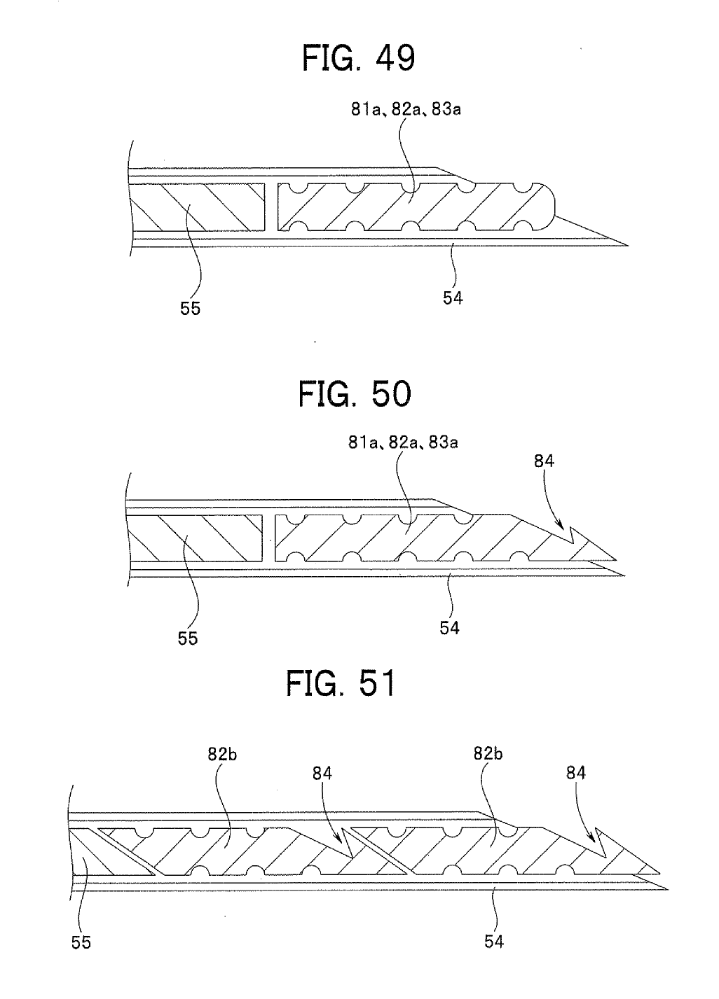

As shown in FIGS. 49 and 50, the ultrasound markers 81a, 82a, and 83a are rod-shaped, and the length is set to a predetermined length. The cross-sectional shape of the ultrasound markers 81a, 82a, and 83a is circular or polygonal, and ultrasound reflection processing, such as concave-convex dimple processing or sandblast processing, is applied to the surfaces.

A barb 84 is arranged at an acicular formation portion of the distal end, which is one end, of the ultrasound markers 81a, 82a, and 83a shown in FIG. 50 to prevent the movement or dropping out of the placed markers.

An ultrasound reflection coating layer containing air bubbles may also be arranged, instead of applying the ultrasound reflection processing on the surfaces of the ultrasound markers 81a, 82a, and 83a.

For example, ultrasound markers 82b, having the same length, are further arranged in the needle tube 54 as shown in FIG. 51.

This allows the operator to insert the needle tube 54 near the organ to place the first ultrasound marker 82b in the organ and to slightly pull back the needle tube 54 to place the second ultrasound marker 82b on the lumen inner wall as an endoscopic marker when delineating the organ serving as a mark. Thus, the placement of the ultrasound marker and the placement of the endoscopic marker can be consecutively performed with one puncture operation. In this case, the marker 82b can be used as an endoscopic marker because the marker 82b can be placed projected halfway from the luminal surface due to the effect of the barb 84.

Arranging at least two ultrasound markers, having the same length, in the needle tube enables to place the makers in the organ and in the lumen inner wall with one type of the ultrasound marker without selectively using the endoscopic marker and the ultrasound marker.

Placing, for example, three types of ultrasound markers 81a, 82a, and 83a with different lengths, two for each, enables to consecutively place the markers 81a, 82a, and 83a in the organ and the lumen inner wall in the movement from the base point to the objective area 10 without pulling out the puncture needle from the treatment instrument channel of the endoscope.

Although both of the endoscopic marker and the ultrasound marker are placed in the above described description, only one of them may be placed.

In the above described procedure for ablating the objective area 10 by the ablation device 3, the ablation margin α is set before the ablation to prevent leaving the diseased tissue. In general, if the thermal denaturation is progressed such that the thermal denaturation of protein can be checked on the ultrasound image, the temperature may be reaching, for example, 100° C., far exceeding the temperature that the cells necrose. If the ablation is performed at 100° C., the surrounding of the cells may also be thermally influenced.

Therefore, the procedure needs to be performed with minimum required heat when ablating an objective area where main blood vessels or nerves are located nearby. It is significantly difficult to ablate without leaving the diseased tissue when performing the procedure with the minimum required heat.

To solve the problem, a temperature sensor can be installed in the ablation device to manage the temperature. However, only the temperature near the center of the ablated part can be managed with the configuration in which the temperature sensor is installed in the ablation device.

Therefore, a procedure is desired in which the functions of the surrounding organs located near the objective area can be preserved under the guidance of ultrasound and the objective area can be surely ablated without leaving the diseased tissue.

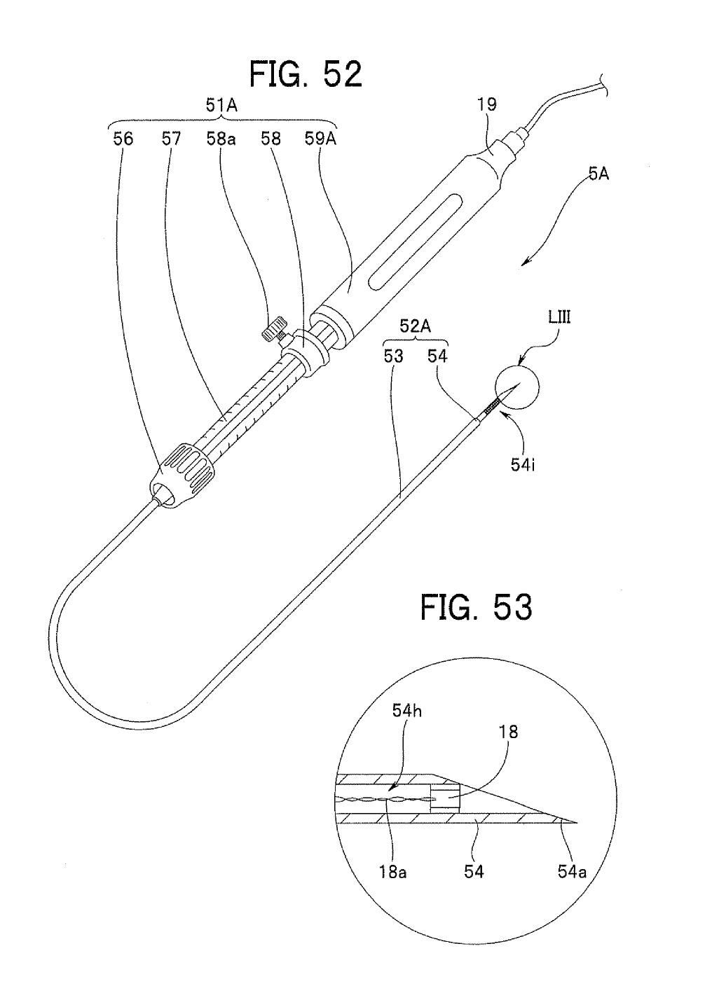

A procedure capable of preserving the functions of the surrounding organs located near the objective area and surely ablating the objective area will be described with reference to FIGS. 52 to 60.

In the ablation method, for example, the EUS 2A1 shown in FIG. 39 that includes a plurality of treatment instrument channels is used as the EUS. A puncture needle 5A with temperature sensor shown in FIGS. 52 to 54 is also used in the procedure.

As shown in FIG. 52, the puncture needle 5A with temperature sensor includes a handle portion 51A and a channel insertion portion 52A. The channel insertion portion 52A includes the sheath 53 and the needle tube 54. Reference numeral 54i denotes an ultrasound reflecting unit. Concave-convex dimple processing or sandblast processing is applied to the surface of the distal end portion of the ultrasound reflecting unit 54i, the distal end portion being projected from the sheath 53.

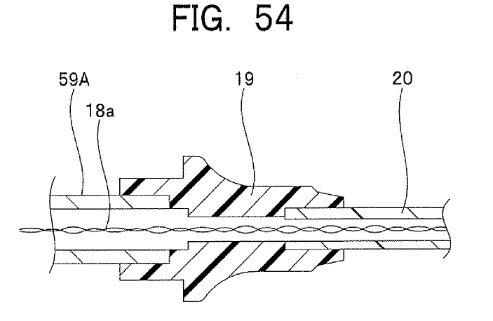

The needle tube 54 is inserted into and slidable with respect to the sheath 53. In the needle tube 54 of the present embodiment, a temperature sensor 18 is arranged at the distal end side in a through hole 54h as shown in FIG. 53. The temperature sensor 18 is fixed to a predetermined location with, for example, an adhesive. A sensor signal line 18a extends from the temperature sensor 18.

The handle portion 51A includes, for example, in order from the distal end, the securing ring 56, the operation portion main body 57, the stopper member 58, and a needle slider 59A.

One end of a sensor cable connection part 19 shown in FIG. 54 is fixedly provided to the proximal end of the needle slider 59A. A sensor cable 20 is fixedly provided to the other end of the sensor cable connection part 19.

One end of the sensor signal line 18a inserted into the sensor cable 20 is connected to the temperature sensor 18 passing through inside the sensor cable connection part 19, the needle slider 59A, and a needle tube 53a. The other end of the sensor signal line 18a is connected to a temperature measuring device not shown.

The temperature measuring device includes a temperature calculating unit and a temperature displaying unit. The temperature calculating unit calculates the temperature based on an outputted signal outputted from the temperature sensor 18 and outputs the calculated temperature to the temperature displaying unit. The temperature displaying unit displays the temperature outputted from the temperature calculating unit.

The temperature measuring device is connected to the power source device 4 and is configured to be able to output, to the power source device 4, a control signal for terminating the output to the ablation device 3 when the measured temperature reaches a predetermined temperature.

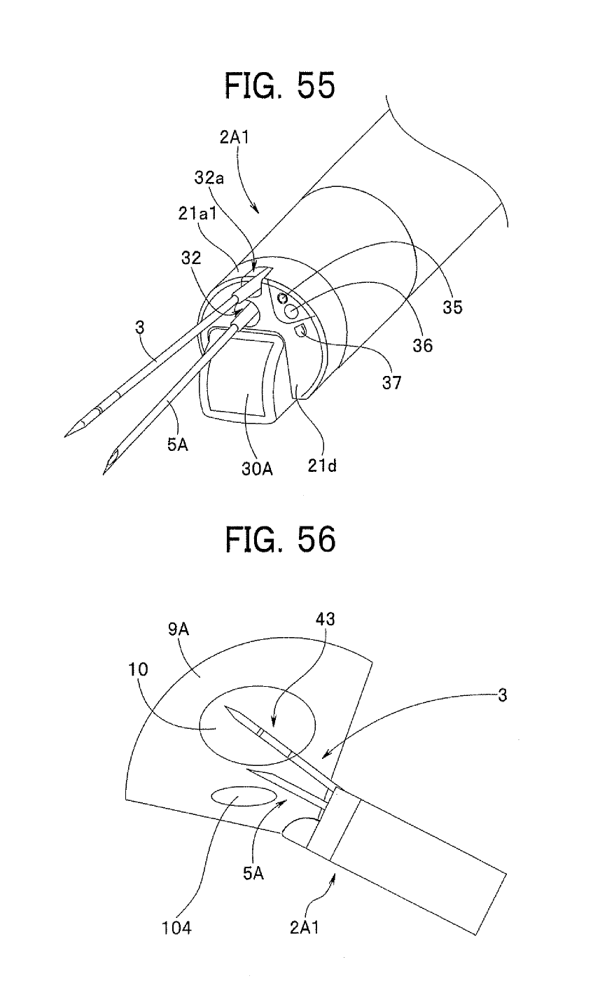

As shown in FIG. 55, for example, the ablation device 3 is projected from the distal end opening 32a, and the puncture needle 5A with temperature sensor is projected from the distal end opening 32 to preserve the functions of the surrounding organs located near the objective area and to surely perform the procedure to ablate the objective area.

Specifically, as shown in FIG. 56, if the objective area 10 and a surrounding organ 104, the function of which is to be preserved, are delineated in the ultrasound image, the operator arranges the electrode portion 43 of the ablation device 3 in the objective area 10. Meanwhile, the operator arranges the needlepoint of the puncture needle 5A with temperature sensor at a desired location between near the surrounding organ 104 and the objective area 10. The ablation of the objective area 10 is then performed.

When performing the ablation, the operator sets the measured temperature of the temperature measuring device to, for example, 60° C. As a result, the temperature measuring device is set to output an output termination signal to the power source device when the temperature sensor 18 detects 60° C.

In the present embodiment, unlike the conventional ablation procedure, the operator can perform the ablation, with the temperature near the surrounding organs being monitored. Therefore, the ablation in the objective area can be raised to a desired temperature. Meanwhile, the thermal influence to the proximity of the surrounding organs can be minimized.

The objective area can be necrosed with minimum required input energy so that the procedure time can be shortened and the energy can be conserved.

The psychological burden of the operator can also be significantly reduced because the temperature near the area, the function of which is to be preserved, is monitored during the ablation and the output of the power source device is terminated when the temperature rises to the preset temperature.

In the present embodiment, the ablation device 3 is projected from the distal end opening 32a, and the puncture needle 5A with temperature sensor is projected from the distal end opening 32. However, the puncture needle 5A with temperature sensor may be projected from the distal end opening 32a and the ablation device 3 may be projected from the distal end opening 32.

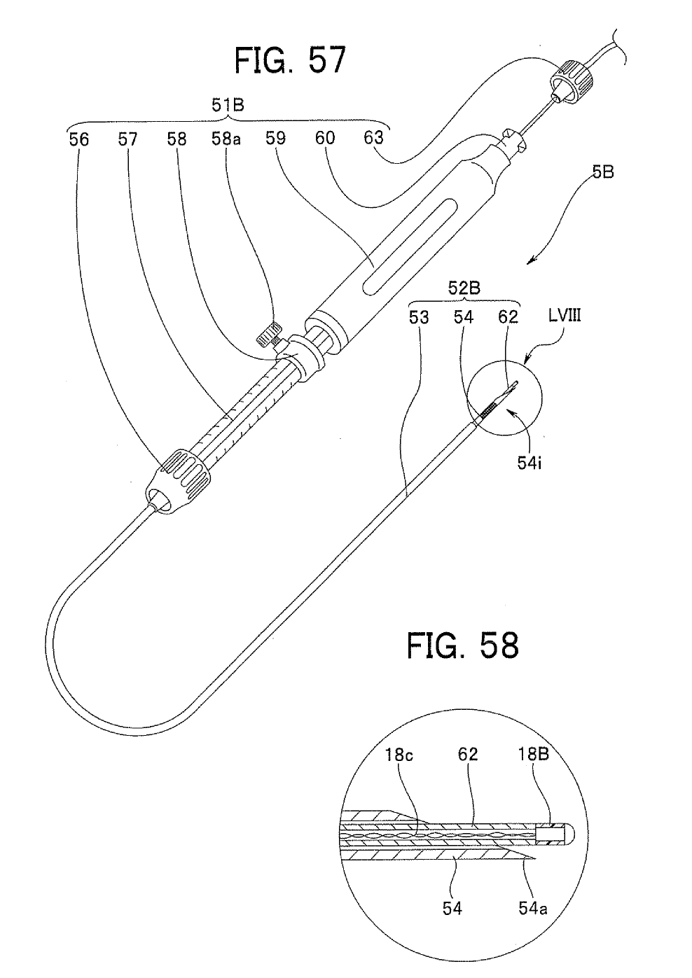

The configuration of the puncture needle with temperature sensor is not limited to the configuration, but may also be configured as shown in FIGS. 57 to 59.

As shown in FIG. 57, a puncture needle 5B with temperature sensor of the present embodiment includes a handle portion 51B and a channel insertion portion 52B. The channel insertion portion 52B includes the sheath 53, the needle tube 54, and a stylet 62.

The stylet 62 is a pipe-shaped insulating portion and is inserted into and slidable with respect to the needle tube 54. As shown in FIG. 58, a temperature sensor 18B is arranged at the distal end of the stylet 62 of the present embodiment. An insulation coating is applied to the temperature sensor 18B. The temperature sensor 18B is fixed by, for example, adhesion. A sensor signal line 18c extends from the temperature sensor 18B.

The handle portion 51A includes, in order from the distal end, the securing ring 56, the operation portion main body 57, the stopper member 58, the needle slider 59, the ferrule portion 60, and a stylet mouthpiece 63.

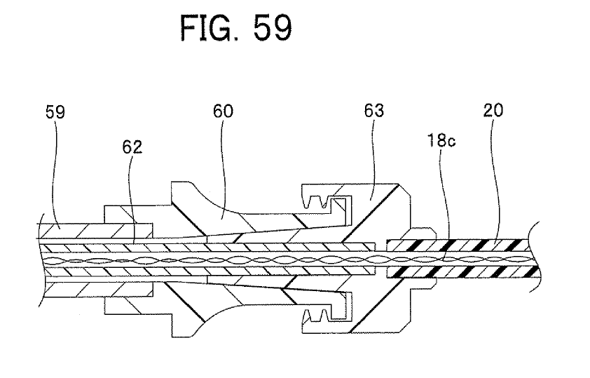

One end of the ferrule portion 60 shown in FIG. 59 is fixedly provided to the proximal end of the needle slider 59. The proximal end portion of the stylet 62 is fixed to one end of the stylet mouthpiece 63, and the sensor cable 20 is fixedly provided to the other end.

One end of the sensor signal line 18c inserted into the sensor cable 20 is connected to the temperature sensor 18B passing through inside the stylet mouthpiece 63 and the stylet 62. The other end of the sensor signal line 18c is connected to the temperature measuring device as in the above described embodiment.

Other configurations are similar to the puncture needle 5A with temperature sensor, and the same members are designated with the same reference numerals and the description will not be repeated.

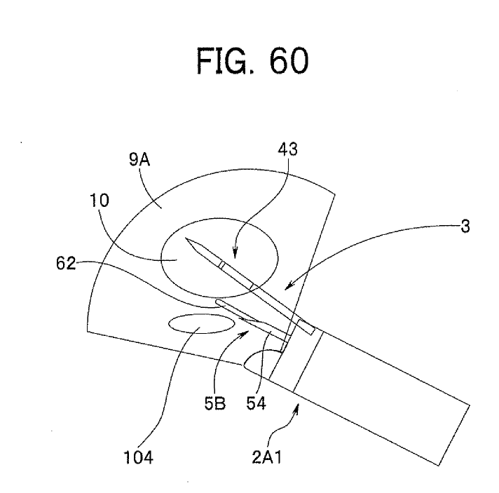

According to the puncture needle 5B with temperature sensor thus configured, if the objective area 10 and the surrounding organ 104, the function of which is to be preserved, are delineated in the ultrasound image as shown in FIG. 60, the operator arranges the electrode portion 43 of the ablation device 3 in the objective area 10. Meanwhile, the needlepoint of the puncture needle 5B with temperature sensor is arranged apart from the electrode portion 43 arranged in the objective area 10. In other words, the temperature sensor 18B that is projecting from the needle tube 54, arranged in the stylet 62 formed of the insulating member, and applied with the insulation coating is arranged at a desired location between near the surrounding organ 104 and the objective area 10. This enables to perform the ablation treatment, with the metal member separated from the ablation device.

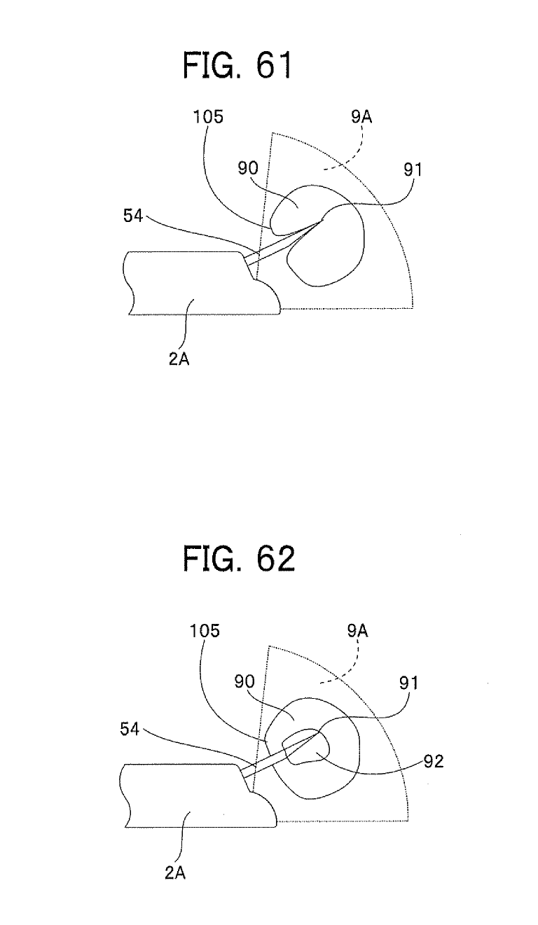

Whether the punctured needlepoint has reached the objective area is determined by the ultrasound image, for example, in the procedure of puncturing the puncture needle in the objective area under the guidance of ultrasound. However, if the objective area is a relatively soft organ such as benign lymph node and gall bladder, even in case where a needlepoint 91 is not penetrated through an outer membrane 105 as shown in FIG. 61, the needlepoint may be delineated as if it has reached the objective area on the ultrasound image. Therefore, a puncture method capable of surely determining on the ultrasound image whether the needlepoint 91 has reached inside the objective area 90 is desired.

The puncture method that enables to determine on the ultrasound image whether the needlepoint has reached inside the objective area will be described with reference to FIGS. 61 to 63.

The operator first punctures the needle tube 54 into the objective area 90 under the guidance of ultrasound as shown in FIG. 61. The operator then supplies an ultrasound contrast agent through the needle tube 54. At this point, an ultrasound contrast agent 92 is injected into the objective area 90 as shown in FIG. 62 if the needle tube 54 has broken through the outer membrane 105 and the needlepoint 91 is located in the objective area 90. Therefore, how the ultrasound contrast agent spreads inside the objective area is delineated in the ultrasound image.

On the other hand, the ultrasound contrast agent 92 is not injected into the objective area 90, but is leaked outside if the needlepoint 91 of FIG. 63 is only significantly transforming the outer membrane 105. Thus, although the ultrasound contrast agent is injected, how the ultrasound contrast agent spreads in the objective area displayed in the ultrasound image is not delineated.

In this way, whether the needlepoint has broken through the outer membrane and reached inside the objective area can be easily and surely determined by injecting the ultrasound contrast agent through the needle tube after puncturing the needle tube.

This enables to accurately determine whether the transition to the next treatment, such as the collection of a specimen or the placement of a guide wire, is possible. Furthermore, an erroneous collection, such as the collection of a specimen other than the objective area, can be surely prevented when collecting the specimen.

The operator can quickly take a measure such as increasing the puncture speed when checking that the needlepoint of the puncture needle has not reached inside the objective area. This can shorten the procedure time and reduce the burden of the operator and the patient.

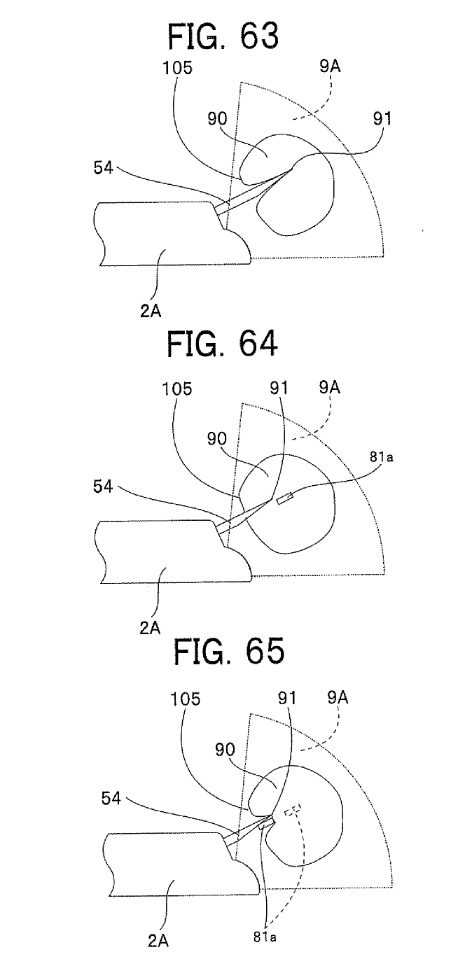

Another puncture method that enables to determine on the ultrasound image whether the needlepoint has reached inside the objective area will be described with reference to FIGS. 61 and 64. In the present embodiment, the ultrasound marker 81a is used, for example, instead of injecting the ultrasound contrast agent for determining whether the needlepoint 91 has reached inside the objective area 90.

The operator first punctures the needle tube 54 in the objective area 90 under the guidance of ultrasound as shown in FIG. 61. In the present embodiment, the operator squeezes the pusher 55 after the puncture and places the ultrasound marker 81a outside the needle tube 54. Subsequently, the operator pulls out the needle tube 54.

At this point, the ultrasound marker 81a is placed in the objective area 90 in spite of the retraction of the needlepoint 91 as shown in FIG. 64 if the needle tube 54 pierces the outer membrane 105 and the needlepoint 91 is located in the objective area 90. Thus, the ultrasound marker 81a arranged inside the objective area is delineated in the ultrasound image.

Meanwhile, when the needlepoint 91 is only significantly transforming the outer membrane 105, after the needle tube 54 is pulled out, the ultrasound marker 81a is moved from a location of a broken line to a location of a solid line as the outer membrane 105 is restored as shown in FIG. 65. Thus, the ultrasound marker 81a located outside the peripheral border of the objective area 90 is delineated in the ultrasound image.

After the puncture, implementing a step of placing the marker enables to easily and surely determine whether the needle point has broken through the outer membrane and reached the objective area.

This enables to obtain effects and advantages that are similar in the case where the ultrasound contrast agent is injected.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A position marker to be placed in a body of a subject, comprising
a main body configured to be inserted and placed in a tissue of the subject; and
an ultrasound reflecting surface formed on a surface of the main body, for reflecting ultrasound.

2. The position marker according to claim 1, wherein the main body has a rod shape.

3. The position marker according to claim 2, wherein a distal end of the rod shape is acicular with a barb.

4. The position marker according to claim 1, wherein the main body is spherical.

5. The position marker according to claim 1, wherein the position marker is absorbed in the living body in the course of time.

6. The position marker according to claim 5, wherein the position marker is made of magnesium alloy.
